SEQUENCE LISTING (12 sequences)

SEQ ID NO: 1 - protein, 5 aa, synthetic construct
SYYMN

SEQ ID NO: 2 - protein, 17 aa, synthetic construct
GISGDPSNTYYADSVKG

SEQ ID NO: 3 - protein, 11 aa, synthetic construct
DLPLVYTGFAY

SEQ ID NO: 4 - protein, 11 aa, synthetic construct
SGDNLRHYYVY

SEQ ID NO: 5 - protein, 7 aa, synthetic construct
GDSKRPS

SEQ ID NO: 6 - protein, 9 aa, synthetic construct
QTYTGGASL

SEQ ID NO: 7 - protein, 120 aa, synthetic construct
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 8 - protein, 109 aa, synthetic construct
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 9 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 10 - DNA, 360 bp, synthetic construct
  source 1..360: mol_type = other DNA
caggtgcaattggtggaaagcggcggcggcctggtgcaaccgggcggcagcctgcgtctgagctgcgcggcctccggatttaccttttcttcttattatatgaattgggtgcgccaagcccctgggaagggtctcgagtgggtgagcggtatctctggtgatcctagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatcttcctcttgtttatactggttttgcttattggggccaaggcaccctggtgacggttagctca

SEQ ID NO: 11 - DNA, 327 bp, synthetic construct
  source 1..327: mol_type = other DNA
gatatcgaactgacccagccgccttcagtgagcgttgcaccaggtcagaccgcgcgtatctcgtgtagcggcgataatcttcgtcattattatgtttattggtaccagcagaaacccgggcaggcgccagttcttgtgatttatggtgattctaagcgtccctcaggcatcccggaacgctttagcggatccaacagcggcaacaccgcgaccctgaccattagcggcactcaggcggaagacgaagcggattattattgccagacttatactggtggtgcttctcttgtgtttggcggcggcacgaagttaaccgttcttggccag

SEQ ID NO: 12 - protein, 10 aa, synthetic construct
GFTFSSYYMN

(19) United States
(12) Patent Application Publication
(10) Pub. No.: US 2024/0343822 A1
(43) Pub. Date: Oct. 17, 2024

(54) TREATMENT OF ANTI-PLA2R AUTOANTIBODY-MEDIATED MEMBRANOUS NEPHROPATHY

(71) Applicant: MORPHOSYS AG, Planegg (DE)

(72) Inventors: Stefan HÄRTLE, Mammendorf (DE); Roland BAUMGARTNER, Planegg/Martinsried (DE)

(21) Appl. No.: 18/580,460

(22) PCT Filed: Jul. 19, 2022
(86) PCT No.: PCT/EP2022/070162

§ 371 Date: Jan. 18, 2024

(30) Foreign Application Priority Data
Jul. 19, 2021 (EP) 21186299.0
Oct. 21, 2021 (EP) 21203870.7

(51) Int. Cl. C07K 16/28; A61K 31/138; A61K 31/167; A61K 31/573; A61K 39/00; A61P 13/12; A61P 37/06

(52) U.S. Cl. CPC C07K 16/2896; A61K 31/138; A61K 31/167; A61K 31/573; A61P 13/12; A61P 37/06; A61K 2039/505; A61K 2039/545

(57) ABSTRACT

The present disclosure relates to the treatment of anti-PLA2R-positive membranous nephropathy. The anti-CD38 antibody, MOR202, is efficacious when administered to patients at certain dosage regimens.

### FIELD OF THE INVENTION

The present disclosure concerns methods for treating anti-PLA2R autoantibody-mediated membranous nephropathy, with an antibody that binds to a CD38 protein. In particular, it relates to an improved dosage regimen of an anti-CD38 antibody, e.g., felzartamab (MOR202), which is safe and effective for treating patients with anti-PLA2R autoantibody-mediated disorders.

### BACKGROUND

Anti-PLA2R autoantibody-mediated membranous nephropathy (aMN), an autoimmune kidney disease, is a primary membranous nephropathy (MN) and the leading cause of nephrotic syndrome in adults worldwide (Ronco P, Debiec H Lancet. 2015 May 16; 385(9981):1983-92). In the US, the incidence of MN is about 1.2 per 100,000; about 3,000 adults are newly diagnosed every year. It typically affects people aged 50-60 years old (Couser W G. Clinical Journal of the American Society of Nephrology. 2017; 12(6):983-97). As many as 40-50% of patients with MN progress to end-stage kidney disease (ESKD) within 10-15 years of diagnosis (Passerini P, et al. Front Immunol 2019; 10(JUN):1-9; Lai W L, et al. J Formos Med Assoc 2015; 114(2):102-11). Even in the absence of ESKD, patients with long-term nephrotic syndrome are at risk of severe and life-threatening thromboembolic events and cardiovascular disease. Around 80% of MN cases are primary and mediated by autoantibodies, while 20% are secondary to other diseases. Approximately 75% of primary MN cases arise due to autoantibodies to the phospholipase A2 receptor (PLA2R) that target PLA2R on podocytes (Bomback A S, et al. Am J Nephrol 2018; 47(suppl 1):30-42). Binding of these autoantibodies induces complement activation and inflammation, which thickens the glomerular membrane and impairs kidney function (Zhang P, et al. J Immunol Res 2021; 1-12). Nephrotic syndrome refers mainly to the presence of heavy proteinuria (loss of protein greater than 3.5 g/day), low serum albumin and marked edema. Although 30-40% of patients may experience spontaneous remission, 30% of patients experience persistent proteinuria with long-term preservation of renal function, and another 30%-50% progress to renal failure within 10-15 years (Trujillo H et al. Portuguese Journal of Nephrology & Hypertension. 2019; 33(1): 19-27). Even if patients with nephrotic syndrome do not progress to renal failure, they have an increased risk of life-threatening thromboembolic and cardiovascular events, and are subject to infections.

M-type phospholipase A2 receptor (PLA2R), a transmembrane protein expressed on podocytes, has been defined as the major autoantigen of MN (Beck L H Jr et al. N Engl J Med. 2009 Jul. 2; 361(1):11-21). Autoantibodies binding to the PLA2R antigen are highly specific to primary MN. Recent investigations revealed the presence of anti-PLA2R autoantibodies in approximately 75% of patients with MN that considerably correlate with disease activity (Bomback A S, Clin J Am Soc Nephrol. 2018 May 7; 13(5):784-786). The fact that the disease-defining glomerular basement changes contain both PLA2R protein as well as antibody deposits provides evidence that anti-PLA2R antibodies play a major causative role in MN. An additional 5% of patients who are negative for anti-PLA2R antibodies have antibodies against another podocyte antigen—the thrombospondin type-1 domain-containing 7A (THSD7A) (Tomas N M et al. N Engl J Med 2014; 371: 2277-2287). Symptoms of MN include, but are not limited to swelling in the legs and ankles, proteinuria, edema, hypoalbuminemia, elevated serum lipids, in particular high cholesterol. Thus, aMN is an immune-mediated glomerular disease characterized by the presence of anti-PLA2R autoantibodies and/or anti-THSD7A autoantibodies.

The main source of anti-PLA2R antibodies are CD38+/CD20− plasma cells and plasmablasts (Halliley J L, et al. Immunity 2015; 43(1):132-45). Higher anti-PLA2R autoantibody titer is associated with more severe disease, longer time to disease remission, and decreased response to anti-CD20 rituximab treatment (Pozdzik A, et al. Biomed Res Int 2018; 6281054; Bech A P, et al. Clin J Am Soc Nephrol 2014; 9(8):1386-925). Therefore, decreasing anti-PLA2R antibody levels and/or preventing the increase of anti-PLA2R antibody levels may prove to be a valid treatment option. So far, however, there is no approved standard treatment for MN. The Kidney Disease Improving Global Outcomes (KDIGO) guidelines recommend using criteria such as anti-PLA2R antibody titer and proteinuria to stratify patients by risk and determine course of treatment (kdigo.org/wp-content/uploads/2017/02/KDIGO-GN-GLPublic-Review-Draft_1-June-2020.pdf Accessed September 2021). The current treatment regimen mainly comprises various non-immunosuppressive drugs (e.g., ACE inhibitors or Angiotensin receptor blockers, statins, and diuretics), conventional immunosuppressive therapies (IST) (e.g., cyclophosphamide combined with steroids, calcineurin inhibitors (CNI), mycophenolat-Mofetil) and off-label use of targeted immunosuppressive drugs (e.g., anti-CD20 antibodies, such as Rituximab) (KDIGO 2020; Ronco P, et al. Journal of Clinical Medicine. 2021; 10(4): 607).

However, these treatments are not effective in all patients and, in particular, conventional ISTs are associated with a high risk of toxicity (Couser W G. Clin J Am Soc Nephrol 2017; 12(6):983-97). A drawback of most immunosuppressive drugs is that they exhibit a considerable degree of toxicity and are associated with significant adverse effects and a high relapse rate. Adverse events comprise infection, infertility, hematologic toxicity, and malignancy later in life. For example, disadvantages of CNIs include long-term nephrotoxicity, the need to closely monitor drug levels and the increased risk for hypertension and diabetes.

In MN, a correlation has been described between the clinical course of the disease and anti-PLA2R autoantibody titer. Patients with a higher anti-PLA2R autoantibody titer have more severe disease and a longer time to disease remission (Pozdzik A, et al. BioMed Research International. 2018:1-19). High titers and sustained or recurrent positivity for anti-PLA2R antibody titers during therapy emerge as negative predictors for outcome (Bomback A S, et al. American Journal of Nephrology. 2018; 47(1):30-42). By contrast, a reduction in anti-PLA2R autoantibody levels is predictive of future likelihood of remission of proteinuria (Ruggenenti P, et al. Journal of the American Society of Nephrology. 2015; 26(10):2545-58).

Due to considerable evidence showing that anti-PLA2R antibodies correlate with disease activity, previously established therapy algorithms are changing.

Patients presenting with high autoantibody titers receive an off-label therapy with anti-CD20 therapeutic antibodies (e.g., rituximab) that allows for a more specific IST by depleting B-cell populations involved as progenitors of plasma cells producing the causative anti-PLA2R autoantibodies. Rituximab response rates seem to be similar to alkylating agents and CNIs, whereas side effects seem to be less than for other drugs used in IST. However, CD20, the target of rituximab is not present on mature long-lived antibody-secreting plasma cells (that are the main source of endogenous immunoglobulins). On early plasmablasts there is only minor residual CD20 expression, compared to the CD20 expression on mature B cells. This is a possible explanation for the sub-optimal efficacy of rituximab therapy in MN patients with high anti-PLA2R antibody titers. As a result, up to 40% of patients failed to respond to anti-CD20 therapies (Bomback, 2018, Couser 2017).

In this respect, direct targeting of plasmablasts as well as plasma cells should lead to a more pronounced reduction in immunoglobulins in general, and, therefore, also to a reduction of autoantibodies. A substantial portion of the anti-PLA2R antibodies in aMN is possibly produced by a long-lived plasma cell pool with a CD20 negative, but CD38 positive immunophenotype, that is not dependent on continuous replenishment of differentiating B-cells. Thus, a direct plasma cell-targeting strategy might have a more profound effect on suppression of pathogenic autoantibodies. In particular, this is important for patients with high levels of autoantibody titers, including those with inadequate response to rituximab (anti-CD20) therapy that maintain high levels of autoantibody titers despite B-cell depletion. As current treatments of aMN are often associated with adverse events and strong side effects, novel therapeutic options and dose administrations are needed for the therapy of aMN.

Felzartamab offers a novel mechanism of action that may have the potential to emerge as an important treatment option for aMN patients compared to current (off-label) treatment options, such as calcineurin inhibitors (CNIs), alkylating cytotoxic drugs (cyclophosphamide) and anti-CD20 antibodies. Felzartamab is an investigational, fully human IgG1 monoclonal anti-CD38 antibody that depletes plasmablasts and plasma cells. Felzartamab specifically binds to the cell surface antigen CD38. Binding of Felzartamab to CD38-positive plasma cells facilitates the depletion of such cells via two modes of action: i) antibody-dependent cell-mediated cytotoxicity (ADCC) in which the plasma cells are lysed by Natural killer (NK) cells, and ii) antibody-dependent cell-mediated phagocytosis (ADCP) in which the macrophages clear away the plasma cells (Endell J, et al. Blood. 2012; 120(21):4018-4018; Raab M S, et al. The Lancet Haematology. 2020; 7(5):e381-e394) (FIG. 10). Its mechanism of action does not involve complement-dependent cytotoxicity, which has been associated with infusion-related reactions (IRRs).

Most therapeutic antibodies are administered by fixed-dosing or body size-adjusted dosing. For the body-size based dosage regimen, mAbs are dosed on body weight or body surface area to equalize mAb exposure between patients.

As a result for the first-in human study of felzartamab MOR202C101 (NCT01421186), the recommended dose in Multiple Myeloma patients was defined at 16 mg/kg (Raab M S, et al. The Lancet Haematology. 2020; 7(5):e381-e394). Based on the pharmacokinetic and pharmacodynamic results of this clinical trial, a modelling and simulation approach was used to identify a suitable dose and dosing regimen for the treatment of aMN patients. The model applied was a minimal physiologically-based PK model (mPBPK model) implementing: i) drug distribution concepts as previously published (Niederalt C, et al. J Pharmacokinet Pharmacodyn 2018; 45(2):235-57; Cao Y, et al. J Pharmacokinet Pharmacodyn 2014; 41(4):375-87); and ii) taking into account that CD38 high-expressing plasma cells as the main target cells are primarily located in tight compartments with limited drug distribution (e.g., the bone marrow). By assuming a certain time range for CD38 turnover, the established mPBPK was able to predict target occupancy rates, which could be compared to in vitro results projecting effective cell killing and therefore clinical efficacy.

Surprisingly, a hybrid dosage regimen of a fixed dose corresponding to a defined body weight range with dose levels similar to 16 mg/kg predicted the best target occupancy rates (Example 2). The finding was unexpected, as compared to MM patients, no tumor cells are present in aMN patients and, therefore, a significantly lower amount of CD38pos cells is present in aMN patients. Nevertheless, due to the limited drug distribution into the tight compartments (e.g., the bone marrow), the dose levels remain similar. The predicted drug levels were best achieved by using fixed dose levels corresponding to a defined body weight range.

Furthermore, the applied treatment intervals are in particular suited for the treatment of aMN, because the higher drug exposure during the initial 4 weeks (weekly or every 2 weeks dosing) is expected to efficiently reduce the number of target cells at the beginning of treatment. Thereafter, no further dosing or a significantly reduced drug exposure is predicted to maintain the reduction of plasma cells over time. The 2 different lengths of exposure as well as a potential retreatment after 3 or 5 months, as described in Example 3, are selected to best characterize the inter-individual variability of the exposure/efficacy relationship considering long term effects and potential effects following retreatment.

Currently, felzartamab is investigated for anti-PLA2R-positive aMN in a phase Ib/IIa trial (M-PLACE; NCT04145440) and a phase IIa trial (New-PLACE; NCT04733040).

Preliminary data indicate that felzartamab is effective in treating anti-PLA2R-positive aMN by eliminating the cells that produce anti-PLA2R autoantibodies. Thereby, felzartamab leads to a reduction of pathologic anti-PLA2R autoantibodies in these patients (e.g., FIGS. 4, 5, 8, 9A,B). Felzartamab shows an effective response in anti-PLA2R-positive aMN patients with unmet medical need, which are not responding or have poor response on existing therapy.

### SUMMARY OF THE INVENTION

The present invention relates to an improved, effective dosage regimen with the anti-CD38 monoclonal antibody, MOR202 (also known as felzartamab), in patients with anti-PLA2R membranous nephropathy.

In an aspect, the invention provides an antibody specific for CD38 comprising an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6) for use in the treatment of a subject with autoantibody-mediated membranous nephropathy, wherein said antibody is administered at a fixed dose level. In some embodiments, the autoantibody-mediated membranous nephropathy is an anti-PLA2R-positive membranous nephropathy. In some embodiments, said antibody is administered at a fixed dose corresponding to a body weight range of the subject. In some embodiments, said antibody is administered at a fixed dose of 650 mg, 975 mg, 1300 mg or 1625 mg, corresponding to a body weight range of <50 kg, 50.5 to 70 kg, 70.5 to 90 kg, >90.5 kg, respectively. In some embodiments, said antibody is administered at a fixed dose of 16 mg/kg. In some embodiments, the antibody is administered in the initial 3 weeks once weekly (QW). In some embodiments, the antibody is administered once every two weeks (q2w). In some embodiments, said antibody is administered at the interval of i) day 1, day 8, day 15, day 29, and day 57 representing a 3 month treatment period or ii) day 1 and day 15 representing a 1 month treatment period. In some embodiments, the antibody for use further comprises a re-treatment 3 or 5 months after end of the preceding treatment period. In some embodiments, the antibody is administered at the interval of day 1, day 8, day 15, and day 22, followed by day 29, day 57, day 85, day 113, and day 141 in a treatment period of 24 weeks. In some embodiments, said antibody is administered at two doses in a treatment interval of 85 days. In some embodiments, said antibody is administered at five doses in a treatment interval of 85 days. In some embodiments, said antibody is administered at nine doses in a treatment interval of 24 weeks. In some embodiments, said antibody is administered at nine doses in a treatment interval of 141 days. In some embodiments, said antibody is administered once weekly during the first month of treatment and once every 4 weeks thereafter. In some embodiments, said antibody is administered intravenously. In some embodiments, the antibody is administered intravenously over a period of two hours. In some embodiments, the subject has a serum level of anti-PLA2R antibodies of ≥50 RU/mL, ≥100 RU/mL, ≥150 RU/mL, ≥200 RU/mL, ≥250 RU/mL, ≥300 RU/mL at screening. In some embodiments, the subject has a serum level of anti-PLA2R antibodies of ≥150 RU/mL. In some embodiments, the subject has a urine protein:creatinine ratio (UCPR; g/g)) of ≥3.0 g/g, ≥4.0 g/g, ≥5.0 g/g, ≥6.0 g/g, or ≥7.0 g/g. In some embodiments, the subject has a proteinuria of ≥3.5 g/24 h, ≥4.0 g/24 h, ≥4.5 g/24 h or ≥5.0 g/24 h from a 24-hour urine screening. In some embodiments, the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100%. In some embodiments, the subject has serum anti-PLA2R antibodies of 50 RU/mL, and the reduction of anti-PLA2R autoantibody titer compared to baseline is >10%, >15%, >20% >25%, >30%, >35%, >40%, >45%, >50%, >55%, >60%, >65%, >70%, >75%, >80%, >85%, >90%, >95%, or 100% at day 8 of cycle 1. In some embodiments, the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100% at day 8 of cycle 1. In some embodiments, the antibody leads to a change from baseline anti-PLA2R titer between 25% and 80%. In some embodiments, the antibody leads to a change from baseline anti-PLA2R titer between 25% and 80% over 4 weeks of treatment. In some embodiments, the antibody leads to a change from baseline anti-PLA2R titer of >80%. In some embodiments, the antibody leads to a change from baseline anti-PLA2R titer of >80% over 4 weeks of treatment. In some embodiments, the antibody does not increase the anti-PLA2R titer over 4 weeks of treatment. In some embodiments, said antibody comprises a variable heavy chain of the sequence QVQLVESGGGLVQPGGSLRLSCAASGFTFSSYYMNWVRQAPGKGLEWVSGISGDPSNTY YADSVKGRFTISRDNSKNTLYLQMNSLRAEDTAVYYCARDLPLVYTGFAYWGQGTLVTVSS (SEQ ID NO: 7) and a variable light chain of the sequence DIELTQPPSVSVAPGQTARISCSGDNLRHYYVYWYQQKPGQAPVLVIYGDSKRPSGIPERFS GSNSGNTATLTISGTQAEDEADYYCQTYTGGASLVFGGGTKLTVLGQ (SEQ ID NO: 8). In some embodiments, said antibody comprises an IgG1 Fc region. In some embodiments, said antibody is formulated for administration in combination with 650 mg to 1,000 mg of oral paracetamol. In some embodiments, said antibody is formulated for administration in combination with 25 mg to 50 mg of oral or intravenous diphenhydramine. In some embodiments, said antibody is formulated for administration in combination with 100 mg of intravenous methylprednisolone, or an equivalent thereof.

In another aspect, the invention concerns a method of treating anti-PLA2R mediated membranous nephropathy in a patient, comprising administering an anti-CD38 antibody to the patient, wherein the anti-CD38 antibody is administered at a fixed dose. Preferably, the anti-CD38 antibody is felzartamab. The antibody is administered at 16 mg/kg once weekly (i.e. 4 doses for cycle 1 in total). In treatment cycles 2-6, felzartamab will be administered at 16 mg/kg once every 4 weeks at the first day of each cycle (i.e., C2D1, C3D1, . . . ; 5 doses for cycles 2-6 in total).

In another aspect, the invention provides felzartamab or a biosimilar thereof for use in the treatment of a subject with anti-PLA2R-mediated membranous nephropathy, wherein felzartamab or a biosimilar thereof is administered at a fixed dose of 650 mg, 975 mg, 1300 mg or 1625 mg, corresponding to a body weight range of <50 kg, 50.5 to 70 kg, 70.5 to 90 kg, >90.5 kg, respectively, and wherein felzartamab or a biosimilar thereof is administered at an interval of i) day 1, day 8, day 15, day 29, and day 57 representing a 3 month treatment period; or ii) day 1 and day 15 representing a 1 month treatment period, and, optionally, with a re-treatment 3 or 5 month after end of the preceding treatment period, and wherein felzartamab or a biosimilar thereof is administered intravenously.

In another aspect, the invention provides a method for treating autoantibody-mediated membranous nephropathy, comprising administering to a subject in need thereof a therapeutically effective amount of an antibody specific for CD38 comprising an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a fixed dose level. In some embodiments, the autoantibody-mediated membranous nephropathy is an anti-PLA2R-positive membranous nephropathy. In some embodiments, said antibody is administered at a fixed dose corresponding to a body weight range of the subject. In some embodiments, said antibody is administered at a fixed dose of 650 mg, 975 mg, 1300 mg or 1625 mg, corresponding to a body weight range of <50 kg, 50.5 to 70 kg, 70.5 to 90 kg, >90.5 kg, respectively. In some embodiments, said antibody is administered at a fixed dose of 16 mg/kg. In some embodiments, the antibody is administered in the initial 3 weeks once weekly (QW). In some embodiments, the antibody is administered once every two weeks (q2w). In some embodiments, said antibody is administered at the interval of i) day 1, day 8, day 15, day 29, and day 57 representing a 3 month treatment period or ii) day 1 and day 15 representing a 1 month treatment period. In some embodiments, the method further comprises re-treatment 3 or 5 months after end of the preceding treatment period. In some embodiments, the antibody is administered at the interval of day 1, day 8, day 15, and day 22, followed by day 29, day 57, day 85, day 113, and day 141 in a treatment period of 24 weeks. In some embodiments, said antibody is administered at two doses in a treatment interval of 85 days. In some embodiments, said antibody is administered at five doses in a treatment interval of 85 days. In some embodiments, said antibody is administered at nine doses in a treatment interval of 24 weeks. In some embodiments, said antibody is administered at nine doses in a treatment interval of 141 days. In some embodiments, said antibody is administered once weekly during the first month of treatment and then once every 4 weeks following the first month. In some embodiments, said antibody is administered intravenously. In some embodiments, the antibody is administered intravenously over a period of two hours. In some embodiments, the subject has a serum level of anti-PLA2R antibodies of ≥50 RU/mL, ≥100 RU/mL, ≥150 RU/mL, ≥200 RU/mL, ≥250 RU/mL, ≥300 RU/mL at screening. In some embodiments, the subject has a serum level of anti-PLA2R antibodies of ≥150 RU/mL. In some embodiments, the subject has a urine protein:creatinine ratio (UCPR; g/g)) of ≥3.0 g/g, ≥4.0 g/g, ≥5.0 g/g, ≥6.0 g/g, or ≥7.0 g/g. In some embodiments, the subject has a proteinuria of ≥3.5 g/24 h, ≥4.0 g/24 h, ≥4.5 g/24 h or ≥5.0 g/24 h from a 24-hour urine screening. In some embodiments, the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100%. In some embodiments, the subject has serum anti-PLA2R antibodies of 50 RU/mL, and the reduction of anti-PLA2R autoantibody titer compared to baseline is >10%, >15%, >20% >25%, >30%, >35%, >40%, >45%, >50%, >55%, >60%, >65%, >70%, >75%, >80%, >85%, >90%, >95%, or 100% at day 8 of cycle 1. In some embodiments, the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100% at day 8 of cycle 1. In some embodiments, the antibody leads to a change from baseline anti-PLA2R titer between 25% and 80%. In some embodiments, the antibody leads to a change from baseline anti-PLA2R titer between 25% and 80% over 4 weeks of treatment. In some embodiments, the antibody leads to a change from baseline anti-PLA2R titer of >80%. In some embodiments, the antibody leads to a change from baseline anti-PLA2R titer of >80% over 4 weeks of treatment. In some embodiments, the antibody does not increase the anti-PLA2R titer over 4 weeks of treatment. In some embodiments, said antibody comprises a variable heavy chain of the sequence QVQLVESGGGLVQPGGSLRLSCAASGFTFSSYYMNWVRQAPGKGLEWVSGISGDPSNTY YADSVKGRFTISRDNSKNTLYLQMNSLRAEDTAVYYCARDLPLVYTGFAYWGQGTLVTVSS (SEQ ID NO: 7) and a variable light chain of the sequence DIELTQPPSVSVAPGQTARISCSGDNLRHYYVYWYQQKPGQAPVLVIYGDSKRPSGIPERFS GSNSGNTATLTISGTQAEDEADYYCQTYTGGASLVFGGGTKLTVLGQ (SEQ ID NO: 8). In some embodiments, said antibody comprises an IgG1 Fc region. In some embodiments, said antibody administered in combination with 650 mg to 1,000 mg of oral paracetamol. In some embodiments, said antibody is administered in combination with 25 mg to 50 mg of oral or intravenous diphenhydramine. In some embodiments, said antibody is administered in combination with 100 mg of intravenous methylprednisolone, or an equivalent thereof.

In another aspect, the invention provides a method for treating anti-PLA2R-mediated membranous nephropathy, comprising administering to a subject in need thereof a therapeutically effective amount of felzartamab or a biosimilar of felzartamab, wherein felzartamab or a biosimilar of felzartamab is administered at a fixed dose of 650 mg, 975 mg, 1300 mg or 1625 mg, corresponding to a body weight range of <50 kg, 50.5 to 70 kg, 70.5 to 90 kg, >90.5 kg, respectively, and wherein felzartamab or a biosimilar of felzartamab is administered at an interval of i) day 1, day 8, day 15, day 29, and day 57 representing a 3 month treatment period; or ii) day 1 and day 15 representing a 1 month treatment period, and optionally with a re-treatment 3 or 5 month after end of the preceding treatment period, and wherein felzartamab or a biosimilar of felzartamab is administered intravenously.

### BRIEF DESCRIPTION OF THE DRAWINGS

FIG. 1 shows schematically the main cell types of B cell differentiation and their level of CD19, CD20 and CD38 expression. The main antibody-secreting cell types targeted by anti-CD38 antibody therapies (e.g., treatment with MOR202) are indicated.

FIG. 2 shows the dosing schedule for the treatment of anti-PLA2R mediated membranous nephropathy (New-PLACE trial, NCT04733040). The treatment phase of the first 3 month, may be followed by a further treatment phase with the same or similar dosing intervals, if no or only a partial immunological response is obtained.

FIG. 3 shows the simulated results for CD38 target occupancy following 5 or 2 doses of felzartamab. Predicted CD38 receptor occupancy results following a fixed dose of felzartamab. Example for an 85 kg aMN patient receiving a dose of 1300 mg. (A) 5 doses; (B) 2 doses; Best case scenario: half-life of CD38 800 min and drug distribution coefficient at 0.85; Worst case scenario: half-life of CD38 80 min and drug distribution coefficient at 0.95.

FIG. 4 shows the relative change of anti-PLA2R antibody titers from baseline 8 weeks after treatment start—Cohort 1 (5-dose schedule) (based on preliminary data from still ongoing New-PLACE trial NCT04733040)

FIG. 5 shows the relative change of anti-PLA2R antibody titers from baseline 8 weeks after treatment start—Cohort 2 (2-dose schedule) (based on preliminary data from still ongoing New-PLACE trial NCT04733040)

FIG. 6 shows the study design of the M-PLACE trial (9-dose schedule) NCT04145440. Anti-PLA2R+: anti-phospholipase-A2 receptor-positive; C: cycle; EOT: end of treatment; FU: follow-up; IST: immunosuppressive treatment; IV: intravenous; MN: membranous nephropathy; TEAE: treatment-emergent adverse event.

FIG. 7 shows the serum concentration of felzartamab during weeks 1-4, pre- and post-dose based on preliminary data from the M-PLACE trial (9-dose schedule) NCT04145440. Following the assessment of felzartamab serum level over the first month of treatment, all patients showed drug exposure in the expected concentration range.

FIG. 8 shows relative change of anti-PLA2R antibody titers from baseline at C1D8, based on preliminary data from the M-PLACE trial (9-dose schedule) NCT04145440. Of 31 patients analyzed, 25 showed a reduction of anti-PLA2R antibody levels after 1 week of treatment, three had an increase, and three did not have Cycle 1 Day 8 data available due to missed visit or early discontinuation. Ab: antibody; anti-PLA2R: anti-phospholipase-A2 receptor; C: cycle; D: day; IgG: immunoglobulin G; IPR: immunologic partial response.

FIG. 9 shows a preliminary phenotypic subgroup analysis based on anti-PLA2R response (≥4 weeks on treatment) based on preliminary data from the M-PLACE trial (9-dose schedule) NCT04145440. FIG. 9A: Deep reduction of anti-PLA2R antibody titer: At last available visit, a change from baseline of >80% reduction, or absolute value <20 U/mL. FIG. 9B: Intermediate reduction of anti-PLA2R antibody titer: At last available visit, a change from baseline between 25-80% reduction (including lower and upper limit). FIG. 9C: Variable anti-PLA2R antibody titer: A patient not meeting criteria of deep reduction or intermediate reduction. If a patient meets the criteria for several categories, they will be classified in the higher category (variable<intermediate, reduction<deep reduction). A surprising finding is that felzartamab is able to induce a rapid and sustained reduction in anti-PLA2R autoantibody titers independent of cohort or initial anti-PLA2R titer, thereby demonstrating proof of mechanism.

FIG. 10 shows a mechanism of action of felzartamab in MN. Plasmablasts and short- and long-lived plasma cells are CD38-positive cells. The anti-CD38 antibody felzartamab leads to a destruction of plasmablasts and plasma cells via ADCC and ADCP and thereby reduces secretion of destructive anti-PLA2R autoantibodies. ADCC: antibody-dependent cell-mediated cytotoxicity; ADCP: antibody-dependent cell-mediated phagocytosis; CD: cluster of differentiation; FcγR: Fc-gamma receptor; NK: natural killer.

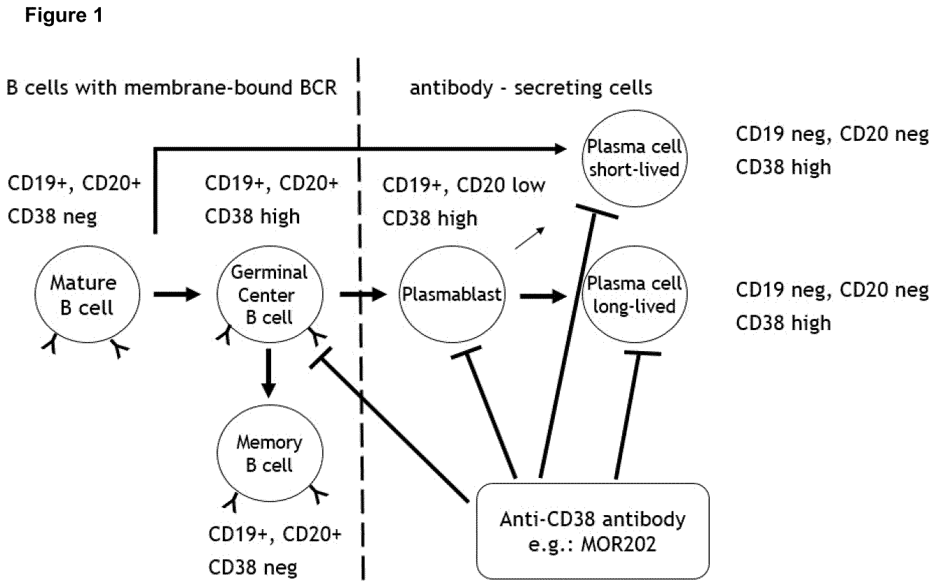

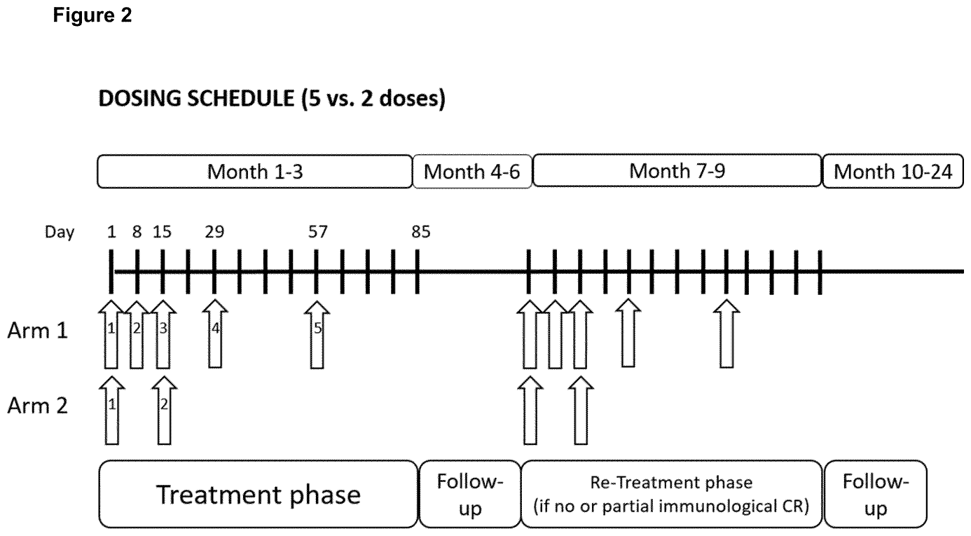

### DETAILED DESCRIPTION OF THE INVENTION

### Definitions

The term “CD38” refers to a protein known as CD38, having the following synonyms: ADP-ribosyl cyclase 1, cADPr hydrolase 1, Cyclic ADP-ribose hydrolase 1, T10.

Human CD38 (UniProt P28907) has the following amino acid sequence:

(SEQ ID NO: 9)
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

CD38 is a type II transmembrane glycoprotein and an example of an antigen that is highly expressed on antibody-secreting cells (including autoantibody-secreting plasmablasts and plasma cells). Functions ascribed to CD38 include both receptor-mediated adhesion, signaling events and (ecto-) enzymatic activity. As an ectoenzyme, CD38 uses NAD+ as substrate for the formation of cyclic ADP-ribose (cADPR) and ADPR, but also of nicotinamide and nicotinic acid-adenine dinucleotide phosphate (NAADP). cADPR and NAADP have been shown to act as second messengers for Ca2+ mobilization. By converting NAD+ to cADPR, CD38 regulates the extracellular NAD+ concentration and, hence, cell survival by modulation of NAD-induced cell death (NCID). In addition to signaling via Ca2+, CD38 signaling occurs via cross-talk with antigen-receptor complexes on T and B cells or other types of receptor complexes, e.g., MHC molecules, and is, in this way, involved in several cellular responses, but also in switching and secretion of IgG antibodies.

The term “antibody” means monoclonal antibodies, including any isotype, such as, IgG, IgM, IgA, IgD and IgE. A preferred isotype of an antibody for use in the present invention is IgG. An IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are referred to as CDR1, CDR2, and CDR3, numbered sequentially from the N-terminus. The more highly conserved portions of the variable regions outside of the CDRs are called the “framework regions”. An “antibody fragment” means an Fv, scFv, dsFv, Fab, Fab′ F(ab′)2 fragment, or other fragment, which contains at least one variable heavy or variable light chain, each containing CDRs and framework regions. Such antibody or antibody fragment may be of any type, such as murine, rat, chimeric, humanized or human antibody or antibody fragment.

“VH” refers to the variable region of an immunoglobulin heavy chain of an antibody or antibody fragment. “VL” refers to the variable region of the immunoglobulin light chain of an antibody or antibody fragment.

The term “anti-CD38 antibody”, as used herein, includes anti-CD38 binding molecules in its broadest sense; any molecule which specifically binds to CD38 or inhibits the activity or function of CD38, or which by any other way exerts an effect on CD38 is included.

Antibodies specific for CD38 are described for example in WO199962526 (Mayo Foundation); WO200206347 (Crucell Holland); US2002164788 (Jonathan Ellis), WO2005103083, WO2006125640 and WO2007042309 (MorphoSys AG); WO2006099875 (Genmab); and WO2008047242 (Sanofi-Aventis). Combinations of antibodies specific for CD38 and other agents are described for example in WO200040265 (Research Development Foundation); WO2006099875 and WO2008037257 (Genmab); and WO2010061360, WO2010061359, WO2010061358 and WO2010061357 (Sanofi Aventis), which are all incorporated by reference in their entireties.

A “human antibody” or “human antibody fragment”, as used herein, is an antibody or antibody fragment having variable regions in which the framework and CDR regions are from sequences of human origin. If the antibody contains a constant region, the constant region also is from such sequences. Human origin includes, but is not limited to human germline sequences, mutated versions of human germline sequences or antibody containing consensus framework sequences derived from human framework sequences analysis, for example, as described in Knappik et al., (2000) J Mol Biol 296:57-86). Human antibodies can be isolated e.g., from synthetic libraries or from transgenic mice (e.g., Xenomouse). An antibody or antibody fragment is human if its sequence is human, irrespective of the species from which the antibody is physically derived, isolated, or manufactured.

The structures and locations of immunoglobulin hypervariable domains, e.g., CDRs, may be defined using well-known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948.

The term “monoclonal antibody” as used herein refers to a preparation of antibody molecules of single molecular composition. A monoclonal antibody composition displays a unique binding site having a unique binding specificity and affinity for particular epitopes.

The present disclosure provides therapeutic methods comprising the administration of a therapeutically effective amount of an anti-CD38 antibody as disclosed to a subject in need of such treatment. A “therapeutically effective amount” or “effective amount”, as used herein, refers to the amount of an antibody specific for CD38, necessary to elicit the desired biological response. In accordance with the subject disclosure, the therapeutic effective amount is the amount of an antibody specific for CD38 necessary to treat and/or prevent autoantibody-mediated membranous nephropathy and symptoms associated with said disorder.

The amount that is effective for a particular therapeutic purpose will depend on the severity of the disease or injury as well as on the weight and general state of the subject. An effective amount for a particular individual may vary depending on factors such as the condition being treated, the overall health of the patient, the method route and dose of administration and the severity of side effects (Maynard, et al. (1996) A Handbook of SOPs for Good Clinical Practice, Interpharm Press; Dent (2001) Good Laboratory and Good Clinical Practice, London, UK).

As used herein, the terms “treat”, “treating”, or the like, mean to alleviate symptoms, eliminate the causation of symptoms either on a temporary or permanent basis, or to prevent or slow the appearance of symptoms of the named disorder or condition.

‘Preventing’ or ‘prevention’ refers to a reduction in risk of acquiring or developing a disease or disorder (i.e., causing at least one of the clinical symptoms of the disease not to develop in a subject that may be exposed to a disease-causing agent, or predisposed to the disease in advance of disease onset. “Prevention” refers to methods which aim to prevent the onset of a disease or its symptoms or which delay the onset of a disease or its symptoms.

The term ‘prophylaxis’ is related to ‘prevention’, and refers to a measure or procedure, the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; the administration of an anti-malarial agent such as chloroquine in advance of a visit to a geographical region where malaria is endemic or the risk of contracting malaria is high.

“Administered” or “administration” includes but is not limited to delivery of a drug by an injectable form, such as an intravenous, intramuscular, intradermal or subcutaneous route or mucosal route, for example, as a nasal spray or aerosol for inhalation or as an ingestible solution, capsule or tablet. Preferably, the administration is by an injectable form. The antibody of the present disclosure can be administered at different time points and the treatment cycle may have a different length. The antibodies may be administered daily, every other day, three times a week, weekly or biweekly. The antibodies may also be administered over at least four weeks, over at least five weeks, over at least six weeks, over at least seven weeks, over at least eight weeks, over at least nine weeks, over at least ten weeks, over at least eleven weeks or over at least twelve weeks.

As used herein, the terms “subject”, “a subject in need thereof” or the like, mean a human or a non-human animal that exhibits one or more symptoms or indicia of autoantibody-mediated membranous nephropathy, and/or who has been diagnosed with autoantibody-mediated membranous nephropathy. Preferably, the subject is a primate, most preferably a human patient who has been diagnosed with autoantibody-mediated membranous nephropathy. Preferably, the autoantibody-mediated membranous nephropathy is anti-PLA2R-positive membranous nephropathy.

“Subject” or “species”, as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject is a primate, most preferably a human.

As used herein, the term “about” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

“Pharmacokinetics” or “PK” as used herein describes how the body affects a specific drug after administration through mechanisms like absorption and distribution, as well as the metabolic changes of the drug in the body, and the effects and routes of excretion of the metabolites of the drug. Pharmacokinetic properties of drugs may be affected by the route of administration and the dose of administered drug.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable vehicle” refers to a diluent, adjuvant, excipient or carrier with which an antibody or antibody fragment is administered.

Throughout this specification, unless the context requires otherwise, the words “comprise”, “have” and “include” and their respective variations such as “comprises”, “comprising”, “has”, “having”, “includes” and “including” will be understood to imply the inclusion of a stated element or integer or group of elements or integers but not the exclusion of any other element or integer or group of elements or integers.

“MOR202” is an anti-CD38 antibody, also known as “MOR03087” or “MOR3087” or “Felzartamab”. The terms are used interchangeable in the present disclosure. MOR202 has an IgG1 Fc region.

The amino acid sequence of the MOR202 HCDR1 according to Kabat is:

| (SEQ ID NO: 1) | |
|---|---|
| | SYYMN |

The amino acid sequence of the MOR202 HCDR2 according to Kabat is:

| (SEQ ID NO: 2) | |
|---|---|
| | GISGDPSNTYYADSVKG |

The amino acid sequence of the MOR202 HCDR3 according to Kabat is:

| (SEQ ID NO: 3) | |
|---|---|
| | DLPLVYTGFAY |

The amino acid sequence of the MOR202 LCDR1 according to Kabat is:

| (SEQ ID NO: 4) | |
|---|---|
| | SGDNLRHYYVY |

The amino acid sequence of the MOR202 LCDR2 according to Kabat is:

| (SEQ ID NO: 5) | |
|---|---|
| | GDSKRPS |

The amino acid sequence of the MOR202 LCDR3 is: QTYTGGASL (SEQ ID NO: 6)

The amino acid sequence of the MOR202 Variable Heavy Domain is:

(SEQ ID NO: 7)
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS

The amino acid sequence of the MOR202 Variable Light Domain is:

(SEQ ID NO: 8)
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ

The DNA sequence encoding the MOR202 Variable Heavy Domain is:

(SEQ ID NO: 10)
CAGGTGCAATTGGTGGAAAGCGGCGGCGGCCTGGTGCAACCGGGCGGCAG
CCTGCGTCTGAGCTGCGCGGCCTCCGGATTTACCTTTTCTTCTTATTATA
TGAATTGGGTGCGCCAAGCCCCTGGGAAGGGTCTCGAGTGGGTGAGCGGT
ATCTCTGGTGATCCTAGCAATACCTATTATGCGGATAGCGTGAAAGGCCG
TTTTACCATTTCACGTGATAATTCGAAAAACACCCTGTATCTGCAAATGA
ACAGCCTGCGTGCGGAAGATACGGCCGTGTATTATTGCGCGCGTGATCTT
CCTCTTGTTTATACTGGTTTTGCTTATTGGGGCCAAGGCACCCTGGTGAC
GGTTAGCTCA.

The DNA sequence encoding the MOR202 Variable Light Domain is:

(SEQ ID NO: 11)
GATATCGAACTGACCCAGCCGCCTTCAGTGAGCGTTGCACCAGGTCAGAC
CGCGCGTATCTCGTGTAGCGGCGATAATCTTCGTCATTATTATGTTTATT
GGTACCAGCAGAAACCCGGGCAGGCGCCAGTTCTTGTGATTTATGGTGAT
TCTAAGCGTCCCTCAGGCATCCCGGAACGCTTTAGCGGATCCAACAGCGG
CAACACCGCGACCCTGACCATTAGCGGCACTCAGGCGGAAGACGAAGCGG
ATTATTATTGCCAGACTTATACTGGTGGTGCTTCTCTTGTGTTTGGCGGC
GGCACGAAGTTAACCGTTCTTGGCCAG.

The amino acid sequence of the MOR202 HCDR1 according to HuCAL is GFTFSSYYMN (SEQ ID NO: 12)

The term “biosimilar” herein is used in a manner consistent with the working definition promulgated by the FDA which defines a biosimilar product to be one that is “highly similar” to a reference product (despite minor differences in clinically inactive components). In practice there can be no clinically meaningful differences between the reference product and the biosimilar product in terms of safety, purity, and potency (Public Health Service (PHS) Act § 262). The “reference product” refers, for example, to commercially available felzartamab.

### Embodiments

The present disclosure relates to an improved dosage regimen for MOR202, an antibody specific for CD38, useful in the prophylaxis and/or treatment of autoantibody-mediated membranous nephropathy, preferably anti-PLA2R-mediated membranous nephropathy or glomerulonephritis.

An antibody specific for CD38 is provided comprising an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6) for use in the treatment of a subject with autoantibody-mediated membranous nephropathy.

The present disclosure also provides pharmaceutical compositions comprising said antibody, and methods for the prophylaxis and/or treatment of autoantibody-positive membranous nephropathy, preferably anti-PLA2R-positive membranous nephropathy or glomerulonephritis, by administering said antibody to the patients under the provided dosage regimen of the invention.

An aspect of the disclosure includes an antibody specific for CD38 comprising an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6) for use in the treatment of autoantibody-mediated membranous nephropathy, wherein said antibody is administered at a fixed dose level.

In a preferred aspect, the antibody is for use in the treatment of anti-PLA2R-positive membranous nephropathy, wherein said antibody is administered at a fixed dose corresponding to a body weight range.

In a further aspect, the antibody for use in the treatment of anti-PLA2R-positive membranous nephropathy is administered at a fixed dose of 650 mg, 975 mg, 1300 mg or 1625 mg, corresponding to a body weight range of <50 kg, 50.5 to 70 kg, 70.5 to 90 kg, >90.5 kg, respectively.

In another embodiment, the antibody for use in the treatment of anti-PLA2R-positive membranous nephropathy is administered in the initial 3 weeks once weekly (QW) or once every two weeks (q2w) at a fixed dose level as indicated above.

In another embodiment, the antibody for use in the treatment of anti-PLA2R-positive membranous nephropathy is administered i) at day 1, day 8, day 15, day 29, day 57; or ii) at day 1 and at day 15 in a treatment interval of 85 days after first administration at a fixed dose level as indicated above.

The antibody for use in the treatment of anti-PLA2R-positive membranous nephropathy is administered at the interval of i) day 1, day 8, day 15, day 29, and day 57, representing a 3 month treatment period or ii) day 1 and day 15, representing a 1 month treatment period.

The antibody for use in the treatment of anti-PLA2R-positive membranous nephropathy is administered at the interval of i) day 1, day 8, day 15, day 29, and day 57 representing a 3 months treatment period or ii) day 1 and day 15 representing a 1 month treatment period, with a re-treatment 3 or 5 month after end of the preceding treatment period.

In another embodiment, the antibody for use in the treatment of anti-PLA2R-positive membranous nephropathy is administered intravenously.

An aspect of the disclosure includes a method of treating anti-PLA2R-positive membranous nephropathy in a subject in need thereof comprising administering to the subject a therapeutically effective amount of an antibody specific for CD38 wherein said antibody comprises an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a dose of about 16 mg/kg or more.

An aspect of the disclosure includes a method of treating anti-PLA2R-positive membranous nephropathy in a subject in need thereof, comprising administering to the subject a therapeutically effective amount of an antibody specific for CD38 wherein said antibody comprises an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a fixed dose corresponding to a body weight range.

An aspect of the disclosure includes a method of treating anti-PLA2R-positive membranous nephropathy in a subject in need thereof, comprising administering to the subject a therapeutically effective amount of an antibody specific for CD38 wherein said antibody comprises an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a fixed dose corresponding to a body weight range. and wherein said antibody is administered at a fixed dose of 650 mg, 975 mg, 1300 mg or 1625 mg, corresponding to a body weight range of <50 kg, 50.5 to 70 kg, 70.5 to 90 kg, >90.5 kg, respectively.

A further aspect includes the use of an antibody specific for CD38 in the manufacture of a medicament for the treatment of anti-PLA2R-positive membranous nephropathy, wherein said antibody comprises an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a dose of about 16 mg/kg or more.

A further aspect includes the use of an antibody specific for CD38 in the manufacture of a medicament for the treatment of anti-PLA2R-positive membranous nephropathy, wherein said antibody comprises an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a fixed dose corresponding to a body weight range.

A further aspect includes the use of an antibody specific for CD38 in the manufacture of a medicament for the treatment of anti-PLA2R-positive membranous nephropathy, wherein said antibody comprises an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a fixed dose corresponding to a body weight range, and wherein said antibody is administered at a fixed dose of 650 mg, 975 mg, 1300 mg or 1625 mg, corresponding to a body weight range of <50 kg, 50.5 to 70 kg, 70.5 to 90 kg, >90.5 kg, respectively.

In certain embodiments, the present disclosure relates to an antibody specific for CD38 for use in the treatment of anti-PLA2R-positive membranous nephropathy, wherein said antibody comprises an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a dose of 16 mg/kg or more.

In an embodiment, the antibody comprises the HCDR1 region of sequence GFTFSSYYMN (SEQ ID NO: 12). In an embodiment, the antibody comprises the HCDR1 region of sequence SYYMN (SEQ ID NO: 1).

In certain embodiments, the disclosed antibody specific for CD38 is administered at a dose of 16 mg/kg or more.

In a preferred aspect, the antibody is for use in the treatment of anti-PLA2R-positive membranous nephropathy, wherein said antibody is administered at 16 mg/kg.

In certain embodiments, the antibody is administered at two doses (i.e. two times) in a treatment interval of 85 days.

In certain embodiments, the antibody is administered at five doses (i.e. five times) in a treatment interval of 85 days.

In certain embodiments, the antibody is administered at nine doses (i.e. nine times) in a treatment interval of 24 weeks.

In certain embodiments, the antibody is administered at nine doses (i.e. nine times) in a treatment interval of 141 days.

In another embodiment the felzartamab is administered once weekly at 16 mg/kg during the first month of treatment and once every 4 weeks thereafter at 16 mg/kg.

In certain embodiments, the antibody is administered intravenously.

In certain embodiments, the antibody is administered intravenously over a period of two hours.

In certain embodiments, the antibody comprises a variable heavy chain of the sequence QVQLVESGGGLVQPGGSLRLSCAASGFTFSSYYMNWVRQAPGKGLEWVSGISGDPSNTY YADSVKGRFTISRDNSKNTLYLQMNSLRAEDTAVYYCARDLPLVYTGFAYWGQGTLVTVSS (SEQ ID NO: 7) and a variable light chain of the sequence DIELTQPPSVSVAPGQTARISCSGDNLRHYYVYWYQQKPGQAPVLVIYGDSKRPSGIPERFS GSNSGNTATLTISGTQAEDEADYYCQTYTGGASLVFGGGTKLTVLGQ (SEQ ID NO: 8).

In certain embodiments, the antibody comprises an IgG1 Fc region.

In a preferred embodiment, the anti-CD38 antibody is felzartamab. In another embodiment, the antibody is a biosimilar of felzartamab.

In another embodiment, the disclosure provides methods for treating and/or prophylaxis of proteinuria associated with anti-PLA2R-positive membranous glomerulonephritis in an individual, which methods comprise the administration of an effective amount of the antibody or antibody fragment, specific for CD38 or one or more of the pharmaceutical compositions herein described.

In another aspect, the disclosure provides methods for preventing the decline of renal function in an individual with anti-PLA2R-positive membranous nephropathy, which methods comprise the administration of an effective amount of the antibody or antibody fragment specific for CD38 or one or more of the pharmaceutical compositions herein described.

In another embodiment, a method for treating anti-PLA2R autoantibody-mediated membranous nephropathy is provided, comprising the steps of:
i) measuring the body weight of a subject with anti-PLA2R-positive membranous glomerulonephritis,
ii) determining the dose of the anti-CD38 antibody to be administered based on the following fixed dose scheme of 650 mg, 975 mg, 1300 mg or 1625 mg, corresponding to a body weight range of <50 kg, 50.5 to 70 kg, 70.5 to 90 kg, >90.5 kg,
iii) administering the antibody with the determined fixed dose to said subject.

In another embodiment, a method for treating anti-PLA2R autoantibody-mediated membranous nephropathy in a subject is provided, comprising the steps of:
i) determining the anti-PLA2R titer in a subject with anti-PLA2R-positive membranous glomerulonephritis before administration of the anti-CD38 antibody,
ii) administering the anti-CD38 antibody felzartamab at a dose of 16 mg/kg at day 1, day 8, day 15, day 22, day 29, day 57, day 85, day 113, and day 141 to the subject, wherein the subject has serum anti-PLA2R antibodies ≥50 RU/mL, ≥100 RU/mL, ≥150 RU/mL, ≥200 RU/mL, ≥250 RU/mL, ≥300 RU/mL (as detected by serum ELISA) at screening.

In one embodiment, the anti-PLA2R titer is determined by serum ELISA.

In another embodiment, felzartamab (MOR202) or a biosimilar of felzartamab is for use in reduction of anti-PLA2R autoantibody titers in patients with anti-PLA2R-positive aMN.

In one embodiment, the antibody with the determined fixed dose is administered in the initial 3 weeks once weekly (QW) or once every two weeks (q2w).

In another embodiment, the antibody is administered with an interval of i) day 1, day 8, day 15, day 29, and day 57; or ii) day 1 and day 15.

In another embodiment, the antibody for use in the treatment of anti-PLA2R-positive membranous nephropathy is administered at the interval of day 1, day 8, day 15, day 22, followed by day 29, day 57, day 85, day 113, day 141 in a treatment period of 24 weeks.

In another embodiment, the antibody for use in the treatment of anti-PLA2R-positive membranous nephropathy is administered at 16 mg/kg. at the interval of day 1, day 8, day 15, day 22, followed by day 29, day 57, day 85, day 113, day 141 in a treatment period of 24 weeks.

In one embodiment, the antibody is for use in the treatment of anti-PLA2R-positive membranous nephropathy, wherein said antibody is administered at 16 mg/kg and wherein the antibody leads to a change from baseline anti-PLA2R titer >10%, >15%, >20% >25%, >30%, >35%, >40%, >45%, >50%, >55%, >60%, >65%, >70%, >75%, >80%, >85%, >90%, >95%, or 100%.

In one embodiment, the antibody is for use in the treatment of anti-PLA2R-positive membranous nephropathy, wherein said antibody is administered at 16 mg/kg and wherein the antibody leads to a change from baseline anti-PLA2R titer >10%, >15%, >20% >25%, >30%, >35%, >40%, >45%, >50%, >55%, >60%, >65%, >70%, >75%, >80%, >85%, >90%, >95%, or 100% at cycle 1, day 8.

In another embodiment, felzartamab (MOR202) or a biosimilar of felzartamab is for use in reduction of anti-PLA2R autoantibody titers in patients with anti-PLA2R-positive aMN, wherein the reduction of anti-PLA2R autoantibody titer compared to baseline is >10%, >15%, >20% >25%, >30%, >35%, >40%, >45%, >50%, >55%, >60%, >65%, >70%, >75%, >80%, >85%, >90%, >95%, or 100% at cycle 1, day 8.

In another embodiment, felzartamab (MOR202) or a biosimilar of felzartamab is for use in reduction of anti-PLA2R autoantibody titers in subjects with anti-PLA2R-positive aMN, wherein the subject has serum anti-PLA2R antibodies >50 RU/mL and wherein the reduction of anti-PLA2R autoantibody titer compared to baseline is >10%, >15%, >20% >25%, >30%, >35%, >40%, >45%, >50%, >55%, >60%, >65%, >70%, >75%, >80%, >85%, >90%, >95%, or 100% at cycle 1, day 8.

In another embodiment, the antibody is for use in the treatment of anti-PLA2R-positive membranous nephropathy, wherein said antibody is administered at 16 mg/kg and wherein the antibody leads to a change from baseline anti-PLA2R titer between 25-80%.

In another embodiment, the antibody is for use in the treatment of anti-PLA2R-positive membranous nephropathy, wherein said antibody is administered at 16 mg/kg and wherein the antibody leads to a change from baseline anti-PLA2R titer between 25-80% over 4 weeks of treatment.

In another embodiment, the antibody is for use in the treatment of anti-PLA2R-positive membranous nephropathy, wherein said antibody is administered at 16 mg/kg and wherein the antibody leads to a change from baseline anti-PLA2R titer >80%.

In another embodiment, the antibody is for use in the treatment of anti-PLA2R-positive membranous nephropathy, wherein said antibody is administered at 16 mg/kg and wherein the antibody leads to a change from baseline anti-PLA2R titer >80% over 4 weeks of treatment.

In another embodiment, the antibody is for use in the treatment of anti-PLA2R-positive membranous nephropathy, wherein said antibody is administered at 16 mg/kg and wherein the antibody stabilizes or maintains the anti-PLA2R titer over 4 weeks of treatment.

In another embodiment, the antibody is for use in the treatment of anti-PLA2R-positive membranous nephropathy, wherein said antibody is administered at 16 mg/kg and wherein the antibody does not increase the anti-PLA2R titer over 4 weeks of treatment.

In one embodiment, felzartamab (MOR202) or a biosimilar of felzartamab is for use as an anti-PLA2R antibody-reducing medicament.

In one embodiment, felzartamab (MOR202) or a biosimilar of felzartamab is for use as an anti-PLA2R antibody titer-stabilizing medicament.

In one embodiment the subjects with anti-PLA2R-positive aMN to be treated have urine protein:creatinine ratio (UPCR; (g/g)) ≥3.0 g/g, ≥4.0 g/g, ≥5.0 g/g, ≥6.0 g/g, or ≥7.0 g/g.

In another embodiment, the subjects with anti-PLA2R-positive aMN to be treated have proteinuria ≥3.5 g/24 h, ≥4.0 g/24 h, ≥4.5 g/24 h or ≥5.0 g/24 h from a 24-hour urine screening.

### WORKING EXAMPLES

### Example 1: Study Population—NewPLACE Study (NCT04733040)

The NewPLACE study is 2-arm, multi-center, open-label, parallel-group phase II trial that will assess the efficacy, safety and pharmacokinetics/pharmacodynamics of the human antibody MOR202 in subjects with anti-PLA2R antibody-positive membranous nephropathy indicated for immunosuppressive therapy.

ClinicalTrials.gov Identifier: NCT04733040—NewPLACE study

Patients were eligible to participate in the study if they met the following criteria:

### Inclusion Criteria

Subjects ≥18 to ≤80 years (at date of signing the informed consent).
Urine protein to creatinine ratio (UPCR) of ≥3.0 g/g (as measured from a 24 hour urine collection) or proteinuria ≥3.5 g/24 h from 24-hour urine at screening.
Estimated glomerular filtration rate (eGFR) ≥50 ml/min/1.73 m². (eGFR >30 and <50 ml/min/1.73 m² can be included provided an interstitial fibrosis and tubular atrophy (IFTA) score of <25% in a kidney biopsy).
Not in spontaneous remission despite proper treatment with angiotensin-converting enzyme inhibitors (ACEI), angiotensin receptor blockers (ARBs) (sufficient dose and treatment duration) as per clinical practice and scientific guidelines. If the subject is intolerant to ACEI and ARBs, the reason must be documented and approval for enrollment be obtained from the Medical Monitor.
Systolic blood pressure (BP) <150 mmHg and diastolic BP <100 mmHg after 5 minutes of rest.
Serum anti-PLA2R antibodies ≥50.0 RU/mL determined by Euroimmun ELISA.
Female subjects: A female is eligible to participate if she is not pregnant, not breastfeeding, and at least one of the following conditions applies:
   a. Not a female of childbearing potential (FCBP), or
   b. a FCBP who agrees to follow the contraceptive guidance during the treatment period and for at least 3 months after last dose of MOR202.

### Key Exclusion Criteria

Hemoglobin <80 g/L (4.96 mmol/L)
Thrombocytopenia: Platelets <100.0×10⁹/L
Neutropenia: Neutrophils <1.5×10⁹/L
Leukopenia: Leukocytes <3.0×10⁹/L
Hypogammaglobulinemia: Serum immunoglobulins ≤4.0 g/L
B-cells <5×10⁶/L
Diabetes mellitus type 2: Subjects with type 2 diabetes mellitus may only enter the clinical trial if a kidney biopsy performed within 6 months prior to screening shows MN without evidence of diabetic nephropathy and diabetes is controlled, as shown by:
Glycated hemoglobin (HbA1c) <8.0% or 64 mmol/mol.
No diabetic retinopathy known.
No peripheral neuropathy known.
Total bilirubin, aspartate aminotransferase or alanine aminotransferase >1.5×ULN, alkaline phosphatase >3.0×ULN.

### Primary Outcome Measures

The primary objective of this study is to assess the efficacy of two different dosing regimens of MOR202 in patients with anti-PLA2R antibody-positive membranous nephropathy characterized by change of anti-PLA2R antibody levels at 3 months compared to baseline.

### Secondary Outcome Measures

To assess the efficacy of two different dosing regimens of MOR202 characterized by anti-PLA2R antibody levels in terms of Immunological Complete Response (ICR) rate at 3 month, 6 months, 12 month and 24 months.

To assess the efficacy of two different dosing regimens of MOR202 in terms of Overall Proteinuria Response (OPR) rate at 6 months, 12 month and 24 months.

To assess the safety of MOR202 as determined by frequency, incidence and severity of treatment-emergent adverse events (TEAEs) through treatment completion, an average of 3 months per treatment period.

To assess the PK profile of MOR202 as determined by serum concentrations over time.

To investigate potential immunogenicity of MOR202 as determined by formation of anti-drug antibody (i.e., number of subjects developing anti-MOR202 antibodies).

### Example 2: Dose Modelling

Previously, a population PK (POP-PK) model was established for felzartamab based on the results of the first-in-human study MOR202C101 in MM patients (NCT01421186; Raab M S, et al. The Lancet Haematology. 2020; 7(5):e381-e394). In order to predict a suitable dose and dosing regimen for the treatment of aMN patients, the existing Pop-PK model was adapted to aMN patients by taking into account the different levels of total CD38 between both populations. It was assumed that aMN subjects have similar CD38 expression levels than healthy volunteers (HV) (expression level=numbers of CD38 molecules per cell type×number of cells). For MM patients, the same amount of CD38 expression was assumed plus the addition of the MM tumour cell population. Values for total CD38 expression were derived from various publications (Brooimans R A et al. Cytometry. Part B, Clinical cytometry 76 (1), S. 18-26; Donohue D M, et al. The Journal of clinical investigation 37 (11), S. 1564-1570; Terstappen L W et al. Blood 76 (9), S. 1739-1747; Muschler G F et al. J. Orthop. Res. 19 (1), S. 117-125; Hernigou P et al. International orthopaedics 37 (11), S. 2279-2287; Fennema E M, et al. Acta orthopaedica 80 (5), S. 618-621; Halliley J L, Tipton C, Liesveld J, et al. Immunity 43 (1), S. 132-145; Loken M R, et al. Cytometry. Part B, Clinical cytometry 76 (1), S. 27-36; Mei H E, et al. Blood 125 (11), S. 1739-1748; Sambuceti G, et al. European journal of nuclear medicine and molecular imaging 39 (8), S. 1326-1338; Trepel F (1974) Klinische Wochenschrift 52 (11), S. 511-515; Hassan et al. Journal of the Royal Society of Medicine 97 (10), S. 465-471).

Based on this evaluation, the following values for total CD38 expression were used leading to a 4.54-fold higher target expression in MM patients compared to aMN patients:
Average CD38 expression level in MM patients: ˜3.5×10\hat{ }16 molecules
Average CD38 expression level in IgAN patients: ˜7.7×10\hat{ }15 molecules

This factor was implemented in the existing POP-PK model from the FiH study MOR202C101 by adjusting the Michaelis-Menten-like non-linear clearance term part of the model.

In a second step, the above-described Pop-PK model for aMN subjects was combined with a minimal physiologically-based PK model (mPBPK model). This combination resulted in a PK/PD modelling approach, which allowed the prediction of felzartamab levels in different organs. The second step was important, as the main target cells of felzartamab (i.e., CD38 high-expressing plasma cells) are primarily located in compartments where monoclonal antibodies only show limited distribution (e.g., immunological niches in the bone marrow).

Considering the main modes of actions of MOR202 (i.e., ADCC and ADCP), CD38 occupancy was identified as a relevant PD parameter for estimating clinical efficacy. As shown by in vitro experiments, felzartamab-induced cell killing was dependent on the number of CD38 molecules expressed on the cell surface of target cells as well as on the number of felzartamab molecules bound per cell (Boxhammer R (2015). MOR202, a Human Anti-CD38 Monoclonal Antibody, Mediates Potent Tumoricidal Activity In Vivo and Shows Synergistic Efficacy in Combination with Different Antineoplastic Compounds. Poster Publication ASH). High cell killing for both modes of actions was observed at approximately ≥150,000 antibodies bound per cell (ABCs), assuming the presence of sufficient and active NK cells or macrophages as effector cells. Lower ABC levels lead to reduced cell-killing effects. Considering a mean CD38 expression rate of approximately 430,000 molecules per plasma cell in the bone marrow, a target occupancy of ˜35% should be achieved to induce relevant cell killing. Nevertheless, higher target occupancy rates are preferred, especially at the beginning of the treatment to account for inter-individual variabilities, like differences in CD38 expression on plasma cells (ranging from about 220,000-780,000 ABCs) or different NK/macrophage cell numbers and cell activities.

In order to evaluate which felzartamab doses would ensure such target occupancy rates, the established PK/PD model was used for simulation, assuming that:
the main target cells of felzartamab (i.e., CD38 high-expressing plasma cells) are primarily located in compartments where monoclonal antibodies only show limited distribution (e.g., the bone marrow); and by
considering a certain time range for CD38 turnover.

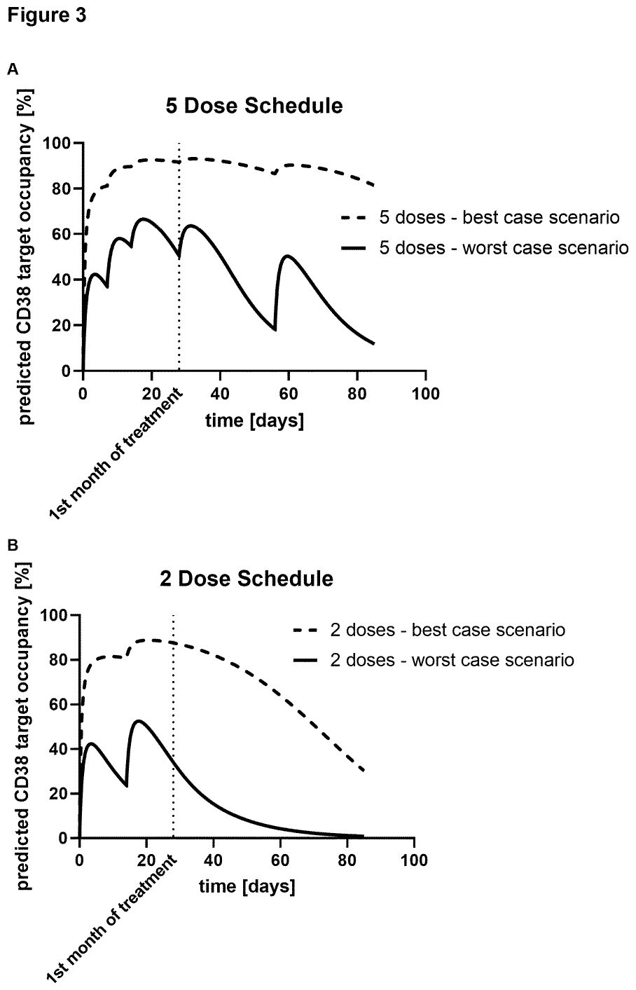

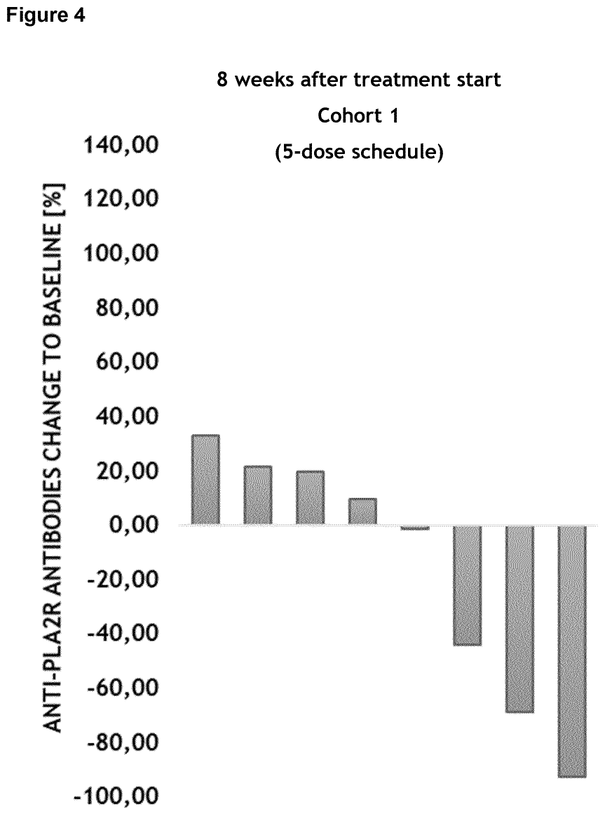

Results for simulating CD38 occupancy for a 85 kg aMN patient with a fixed dose of 1300 mg felzartamab are summarize in FIG. 3, considering a 5 and 2 dose schedule. Applying 5 doses of felzartamab, target occupancy rates between 81% and 92% were predicted over the first month of treatment for the best case simulation scenario (half-life of CD38 at 800 min and drug distribution coefficient at 0.85). For the worst case simulation scenario, target occupancy rates between 37% and 54% were predicted over the first month of treatment (half-life of CD38 at 80 min and drug distribution coefficient at 0.95). Applying 2 doses of felzartamab, target occupancy rates were predicted between 81% and 88% for the best case scenario and between 24% and 52% for the worst case scenario. Similar results were also observed for the other body weight ranges as shown in Example 3.

### Example 3: NewPLACE—Dosing

MOR202 will be dosed depending on patient body weight. Body weight is to be measured either the day before or preferably the day of infusion. Four fixed dose levels will be used corresponding to 4 body weight ranges:

TABLE 1
The absolute dose of MOR202 to be
administered intravenously (iv) is:

| Body weight [kg] | ≤50 | >50 to 70 | >70 to 90 | >90 |
|---|---|---|---|---|
| MOR202 dose [mg] | 650 | 975 | 1300 | 1625 |
| Number of MOR202 vials | 2 | 3 | 4 | 5 |

### M1 Arm:

5 doses of MOR202 administered on Day 1, 8, 15, 29 and 57

### M2 Arm:

2 doses of MOR202 administered on Day 1 and 15

Subjects in the M1 arm without ICR in anti-PLA2R antibody levels at 6 month (Day 183) will receive the same course of therapy starting on Day 204 (5 doses). Subjects in the M2 arm with Immune Partial Response (IPR) will receive the same course of therapy starting on Day 204 (2 doses). Subjects in the M2 arm without ICR and without IPR in anti-PLA2R antibody levels at 6 month (Day 183) will receive a 5-dose therapy starting on Day 204 (5 doses). Thus, subjects will receive 650 mg to 1625 mg MOR202 per dose (intravenously; IV) in 5 doses administered on Day 1, Day 8, Day 15, Day 29, and Day 57; or 2 doses administered on Day 1 and Day 15.

Medications to reduce the risk of infusion-related reactions (IRRs) with MOR202 should be administered 30 to 60 minutes prior to infusion:
Oral paracetamol 650 to 1,000 mg
Oral or intravenous diphenhydramine 25 to 50 mg or equivalent
Methylprednisolone 100 mg, or equivalent, administered intravenously

The first infusion of MOR202 shall last approximately 90 minutes. If no infusion reactions occur, the infusion time may be shortened to 1 hour for the 2nd administration or shorter in subsequent infusions. Infusion time should not be shorter than 30 minutes.

TABLE 2
Recommendation for MOR202 infusion speed and premedication

| | | | | 4th and |
|---|---|---|---|---|
| MOR202 infusion number | 1 | 2 | 3 | onwards |
| Recommended maximum | 2.5 | 4 | 6 | 8 |
| infusion speed (mL/min) | | | | |
| based on 250 ml volume | | | | |
| Methylprednisolone IV or | 100 mg | 100 mg | 50 mg | not |
| equivalent (mg) | | | | mandatory |

Subjects may be treated again at day 204 in case no complete response in anti-PLA2R antibody levels is observed at the 6-month visit (i.e., day 183).

### Example 4: Preliminary Results New-PLACE (NCT04733040)

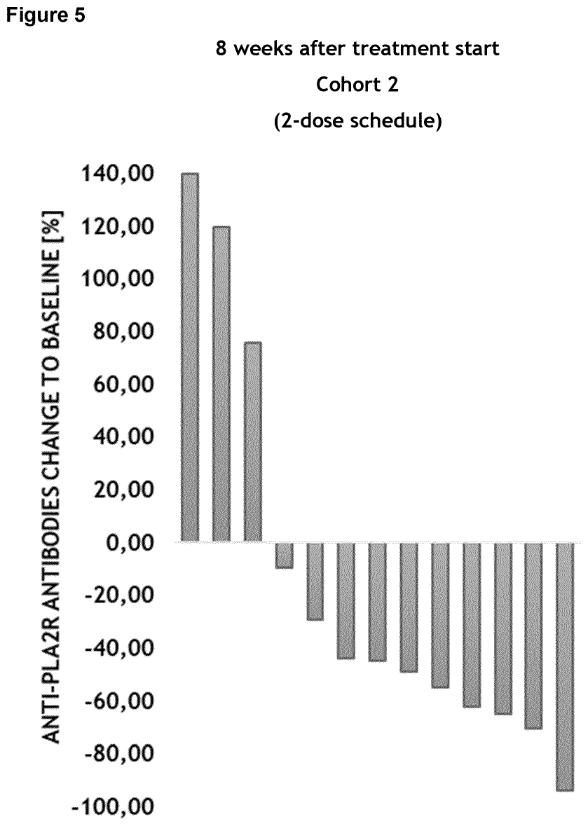

A preliminary assessment on anti-PLA2R antibody levels 8 weeks after treatment start with felzartamab are shown in FIG. 4 (cohort M1, 5-dose schedule) and FIG. 5 (cohort M2—2 dose schedule). For most of the patients a reduction of anti-PLA2R antibody levels after 8 weeks of treatment was observed. In the few patients of cohort M1 (5-dose schedule) (FIG. 4) where no reduction of anti-PLA2R antibody levels after 8 week of treatment was observed, there was no strong increase of autoantibody levels. Most patients of cohort M2 (2-dose schedule) (FIG. 5) responded with a deep decrease of anti-PLA2R antibody levels after 8 weeks of treatment. The increase of autoantibody levels in the few patients where no drop of autoantibodies occurred in the cohort M2 (2-dose schedule) appeared to be more pronounced as for corresponding patients of cohort M1 (5-dose schedule).

Overall, the observed reduction of anti-PLA2R antibody titer after 8 weeks of treatment is independent of cohort and suggests a successful depletion of CD38+ plasma cells in most of the patients treated with felzartamab.

### Example 5: Preliminary Results M-PLACE (NCT04145440)

Enrolled patients were aged 18-80 years with biopsy-proven anti-PLA2R-positive MN requiring IST according to the site investigator. Other key inclusion criteria were:
Urine protein:creatinine ratio (UPCR) ≥3.0 g/g or proteinuria ≥3.5 g/24 h from a 24-hour urine screening
Estimated glomerular filtration rate (eGFR) ≥50 mL/min/1.73 m² or eGFR ≥30 and <50 mL/min/1.73 m² with interstitial fibrosis and tubular atrophy score of <25% in a renal biopsy

A total of 31 patients were enrolled into one of two cohorts:
Cohort 1: newly diagnosed patients (Cohort 1A) and those who relapsed on prior therapy (Cohort 1B); patients have serum anti-PLA2R antibodies ≥50 RU/mL (central laboratory, Euroimmun ELISA)
Cohort 2: patients with anti-PLA2R-positive MN refractory to a prior IST and requiring another IST

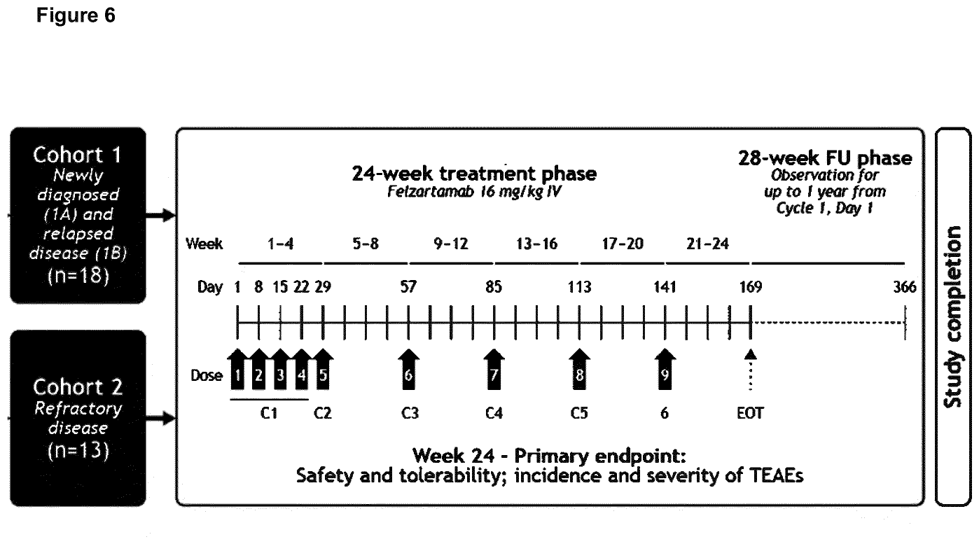

Patients received 9 doses of felzartamab 16 mg/kg intravenously (IV), once weekly during the first month of treatment and once every 4 weeks thereafter (FIG. 6). After the end of treatment, patients remained in follow-up for up to 28 weeks.

The primary endpoint was the incidence and severity of treatment-emergent adverse events (TEAEs). TEAEs were graded according to the National Cancer Institute Common Terminology Criteria for Adverse Events, version 5.0.

Key secondary endpoints were best immunologic response, rate of immunologic complete response (ICR), and immunologic partial response (IPR) based on reduction of serum anti-PLA2R antibody titer:
ICR was defined as a reduction in anti-PLA2R antibody titer to <14 RU/mL
IPR was defined as a >50% reduction in anti-PLA2R antibody titer from baseline

Pharmacokinetics (PK; felzartamab serum levels) and immunogenicity (anti-drug antibodies) were assessed at selected time points for all patients during the course of the study. A model-based prediction for PK was performed before study start based on PK data from the first human study (NCT01421186) and expected total CD38 expression rates in patients with MN.

In a safety run-in period the first three patients were treated and observed until Cycle 2, Day 1, or until they demonstrated a toxicity leading to discontinuation of treatment. Treatment initiation of further patients was paused until completion of the safety run-in period, and patients involved in the safety run-in continued the trial as per protocol.

### Preliminary Results (M-PLACE):

31 patients had been enrolled (Cohort 1A: 15 patients; Cohort 1B: 3 patients; Cohort 2: 13 patients).

TABLE 1
Characteristics and disposition for patients enrolled in the study.

| | | Cohort 1A | Cohort 1B | Cohort 2 | |
|---|---|---|---|---|---|
| | | Newly diagnosed | Relapsed | Refractory | Total |
| | | (n = 15) | (n = 3) | (n = 13) | (n = 31) |
| Demographics | | | | | |
| Gender | Male, n (%) | 10 (66.7) | 3 (100) | 11 (84.6) | 24 (77.4) |
| Age (at screening), years | Median | 62.0 | 61.0 | 59.0 | 61.0 |
| | SD | 12.19 | 5.69 | 12.47 | 11.81 |
| | Min, Max | 33, 77 | 58, 69 | 28, 71 | 28, 77 |
| Baseline anti-PLA2R*, U/mL | Mean | 216.4 | 165.3 | 301.5 | 247.1 |
| | SD | 127.72 | 52.63 | 377.57 | 259.31 |
| | Min, Max | 67, 514 | 115, 220 | 10, 1075 | 10, 1075 |
| Time since initial MN diagnosis | Mean | 4.38 | 126.67 | 37.61 | 30.15 |
| from screening, months | | | | | |
| | SD | 3.48 | 96.30 | 41.57 | 51.02 |
| | Min, Max | 0.43, 11.86 | 33.68, 225.97 | 10.41, 169.17 | 0.43, 225.97 |

*Baseline anti-PLA2R is the last value prior to treatment start.

TABLE 2
Patient disposition.

| | | Cohort 1A | Cohort 1B | Cohort 2 | |
|---|---|---|---|---|---|
| | | Newly diagnosed | Relapsed | Refractory | Total |
| | | (n = 15) | (n = 3) | (n = 13) | (n = 31) |
| Patient disposition | | | | | |
| ≥1 week of treatment completed | n (%) | 14 (93.3) | 3 (100) | 11 (84.6) | 28 (90.3) |
| ≥4 weeks of treatment completed | n (%) | 14 (93.3) | 3 (100) | 10 (76.9) | 27 (87.1) |
| ≥12 weeks of treatment completed | n (%) | 6 (40.0) | 2 (66.7) | 9 (69.2) | 17 (54.8) |
| Entered Follow-up phase | n (%) | 5 (33.3) | 1 (33.3) | 3 (23.1) | 9 (29.0) |

### Safety

The adverse events (AEs) occurring during the safety run-in were mostly mild in severity, and the study could continue as per protocol. The safety profile was consistent with the mechanism of action of felzartamab and AEs were manageable. Of 31 patients, 26 (84%) experienced 164 TEAEs. Five of 31 patients (16%) experienced five treatment-emergent serious AEs (SAEs): two events were considered related to felzartamab (one type-I hypersensitivity and one IRR), and three events were not related to felzartamab. No event had a fatal outcome. Seven of 31 patients (23%) experienced ten treatment-emergent AEs of special interest (AESIs), the majority being allergic reactions to felzartamab (seven events in four patients). Other AESIs were one grade 3 neutropenia, one grade 3 IRR and one grade 4 COVID-19 (considered not related to felzartamab). Four of 31 patients (13%) discontinued felzartamab treatment due to AEs: one grade 3 chest pain, one grade 3 type-I hypersensitivity, one grade 4 COVID-19 and one grade 3 IRR. All events were considered related to felzartamab, with the exception of COVID-19.

### Pharmacokinetics and Immunogenicity

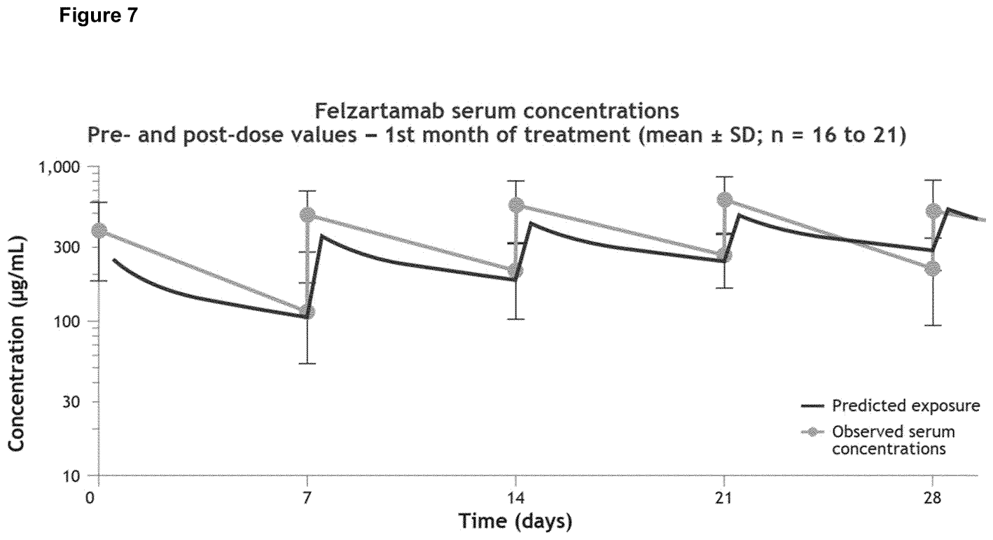

Following the assessment of felzartamab serum level over the first month of treatment, all patients showed drug exposure in the expected concentration range (FIG. 7). Anti-drug antibodies (ADAs) were analyzed in 21 of 31 patients. Two patients had preexisting ADAs with no observed impact on felzartamab PK following the first administration; no treatment-boosted or treatment-emergent ADAs were detected.

### Immunologic Responses

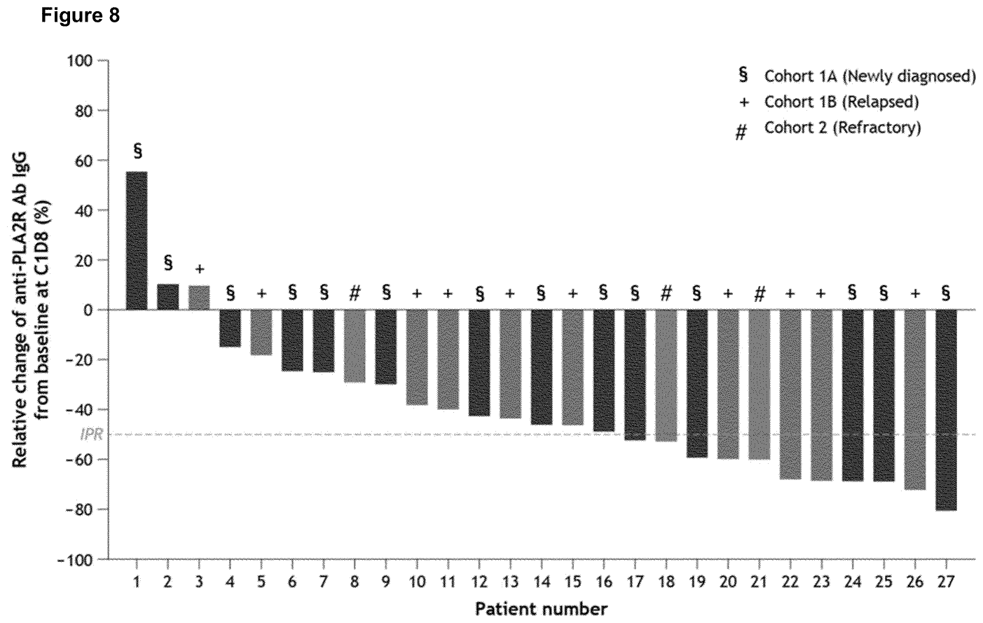

Of the 31 patients enrolled, 25 showed an initial reduction of anti-PLA2R antibody levels after 1 week of treatment, three had an increase, and three did not have Cycle 1 Day 8 data available due to a missed visit or early discontinuation (FIG. 8). Almost all patients experienced an immunologic response by Cycle 1 Day 8. Felzartamab rapidly and substantially reduced anti-PLA2R antibody titers in patients with anti-PLA2R-positive MN.

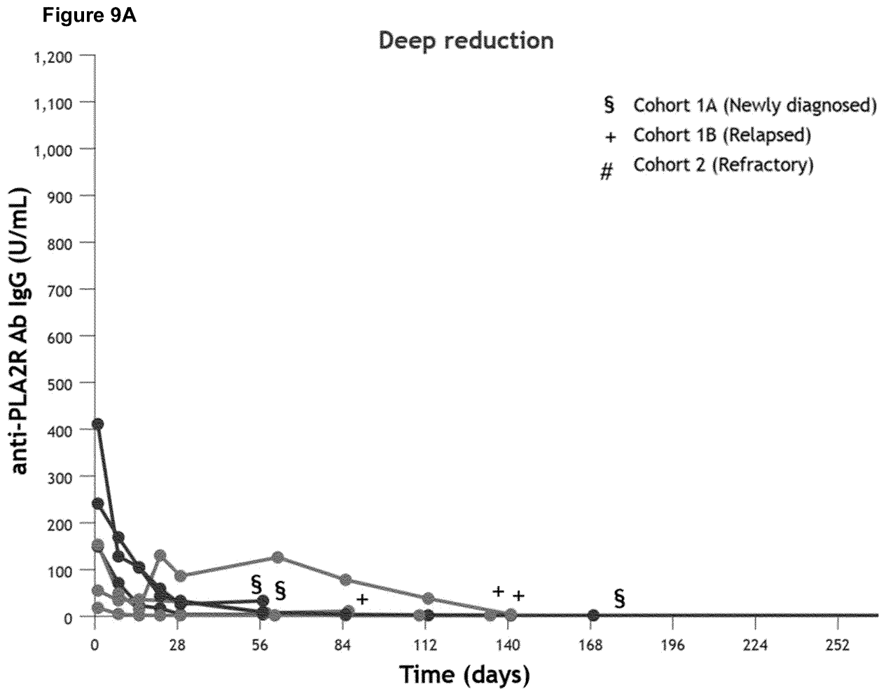

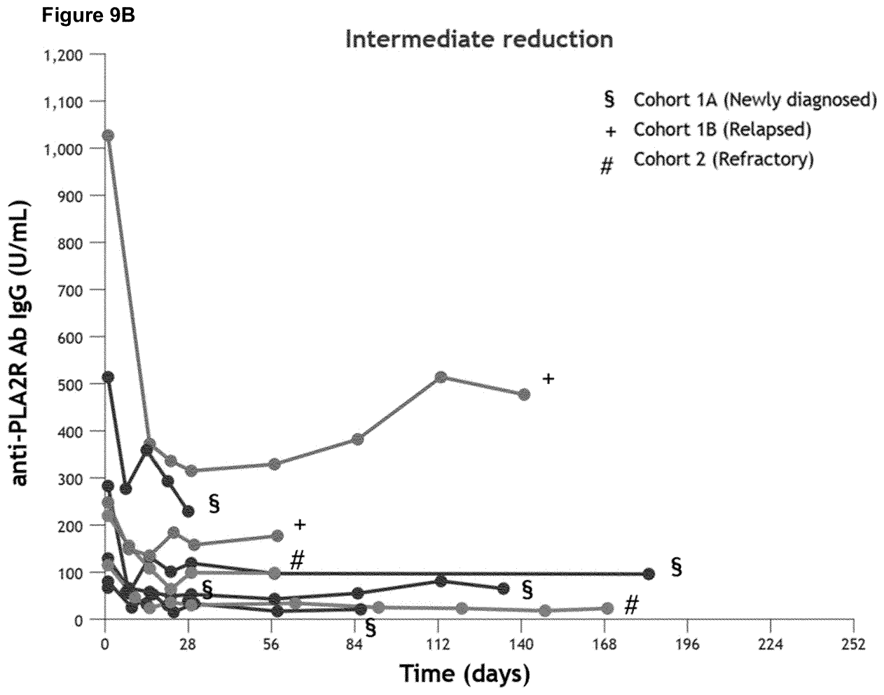

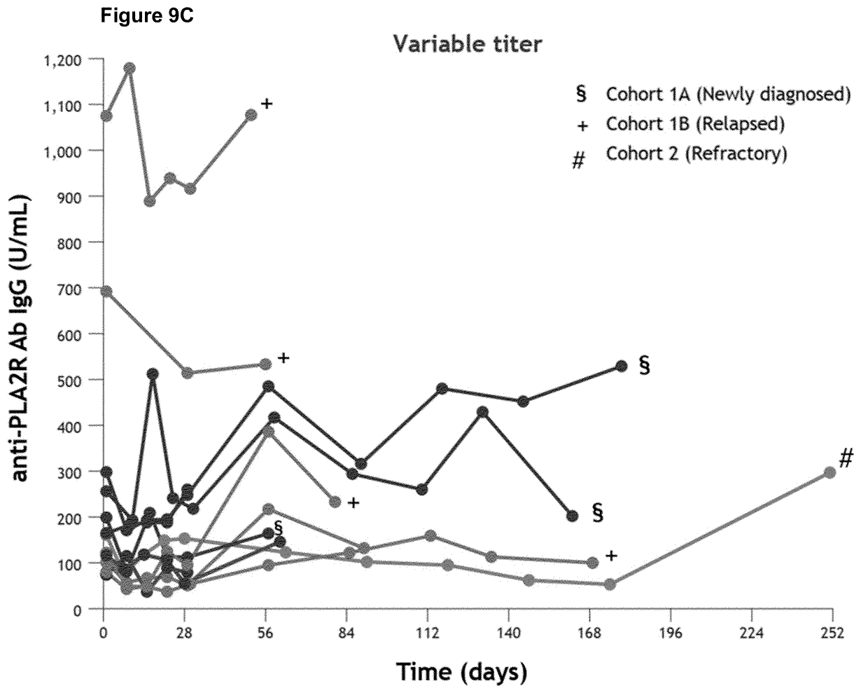

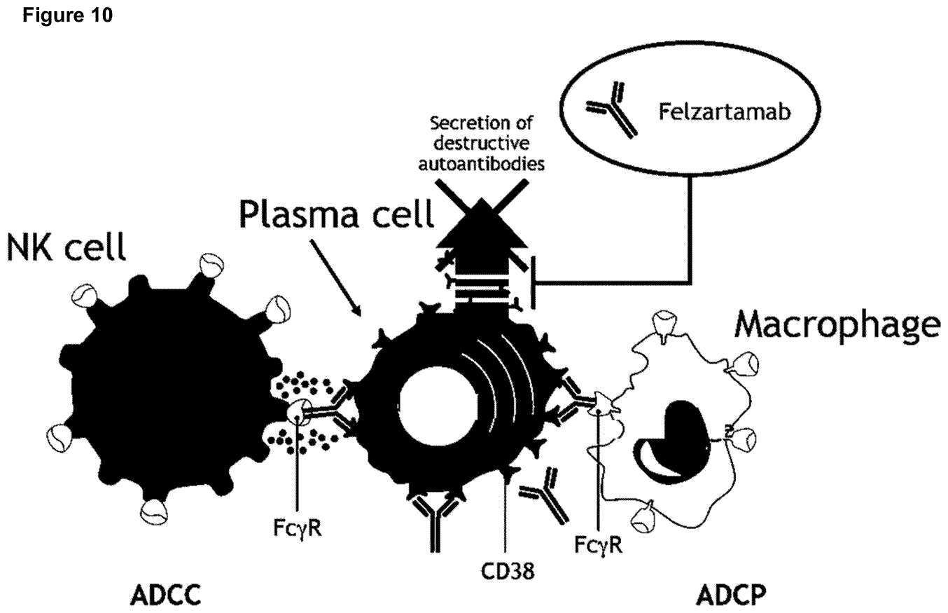

Based on long-term (≥4 weeks) anti-PLA2R antibody kinetics, three subgroups were identified across both cohorts, (FIG. 9):
Patients with a continuous and deep reduction in anti-PLA2R titer: a change from baseline of >80% reduction, or absolute value <20 U/mL at last available visit (n=6)
Patients with an intermediate reduction in anti-PLA2R titer: a change from baseline between 25-80% reduction at last available visit (n=9)
Patients with variable anti-PLA2R titer: not meeting criteria of deep reduction or intermediate reduction (n=14). The titer in these patients appeared to be in a constant range over time, with no continuous increase of autoantibody levels.

Although data are preliminary, an initial rapid reduction in anti-PLA2R antibody titer was observed in most patients 1 week after the first felzartamab administration; the overall degree of response was heterogenous, as expected in patients with MN (Gu Y, et al. Biomolecules 2021; 11(4):513). The observed reduction of anti-PLA2R antibody titer was independent of cohort and suggests a successful depletion of CD38+ plasma cells.

Following an early assessment 6 months after treatment initiation, the decrease in anti-PLA2R antibody titer was associated with a decrease of UPCR in the majority of patients, further supporting the potential role of anti-PLA2R antibody levels on clinical response.

1. An antibody specific for CD38 comprising an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6) for use in the treatment of a subject with autoantibody-mediated membranous nephropathy, wherein said antibody is administered at a fixed dose level.

2. The antibody for use according to claim 1, wherein the autoantibody-mediated membranous nephropathy is an anti-PLA2R-positive membranous nephropathy.

3. The antibody for use according to claim 1 or claim 2, wherein said antibody is administered at a fixed dose corresponding to a body weight range of the subject.

4. The antibody for use according to claim 3, wherein said antibody is administered at a fixed dose of 650 mg, 975 mg, 1300 mg or 1625 mg, corresponding to a body weight range of <50 kg, 50.5 to 70 kg, 70.5 to 90 kg, >90.5 kg, respectively.

5. The antibody for use according to claim 1 or 2, wherein said antibody is administered at a fixed dose of 16 mg/kg.

6. The antibody for use according to any of the preceding claims, wherein the antibody is administered in the initial 3 weeks once weekly (QW).

7. The antibody for use according to any of the preceding claims, wherein the antibody is administered once every two weeks (q2w).

8. The antibody for use according to any of the preceding claims, wherein said antibody is administered at the interval of i) day 1, day 8, day 15, day 29, and day 57 representing a 3 month treatment period or ii) day 1 and day 15 representing a 1 month treatment period.

9. The antibody for use according to claim 8, further comprising re-treatment 3 or 5 months after end of the preceding treatment period.

10. The antibody for use according to any one of claims 1-5, wherein the antibody is administered at the interval of day 1, day 8, day 15, and day 22, followed by day 29, day 57, day 85, day 113, and day 141 in a treatment period of 24 weeks.

11. The antibody for use according to any one of claims 1-5, wherein said antibody is administered at two doses in a treatment interval of 85 days.

12. The antibody for use according to any one of claims 1-5, wherein said antibody is administered at five doses in a treatment interval of 85 days.

13. The antibody for use according to any one of claims 1-5, wherein said antibody is administered at nine doses in a treatment interval of 24 weeks.

14. The antibody for use according to any one of claims 1-5, wherein said antibody is administered at nine doses in a treatment interval of 141 days.

15. The antibody for use according to claim 5, wherein said antibody is administered once weekly during the first month of treatment and then once every 4 weeks following the first month.

16. The antibody for use according to any of the preceding claims, wherein said antibody is administered intravenously.

17. The antibody for use according to claim 16, wherein the antibody is administered intravenously over a period of two hours.

18. The antibody for use according to any of the preceding claims, wherein the subject has a serum level of anti-PLA2R antibodies of ≥50 RU/mL, ≥100 RU/mL, ≥150 RU/mL, ≥200 RU/mL, ≥250 RU/mL, ≥300 RU/mL at screening.

19. The antibody for use according to claim 18, wherein the subject has a serum level of anti-PLA2R antibodies of ≥150 RU/mL.

20. The antibody for use according to any of the preceding claims, wherein the subject has a urine protein:creatinine ratio (UCPR; g/g)) of ≥3.0 g/g, ≥4.0 g/g, ≥5.0 g/g, ≥6.0 g/g, or ≥7.0 g/g.

21. The antibody for use according to any of the preceding claims, wherein the subject has a proteinuria of ≥3.5 g/24 h, ≥4.0 g/24 h, ≥4.5 g/24 h or ≥5.0 g/24 h from a 24-hour urine screening.

22. The antibody for use according to any of the preceding claims, wherein the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100%.

23. The antibody for use according to claim 22, wherein the subject has serum anti-PLA2R antibodies of ≥50 RU/mL, and the reduction of anti-PLA2R autoantibody titer compared to baseline is >10%, >15%, >20% >25%, >30%, >35%, >40%, >45%, >50%, >55%, >60%, >65%, >70%, >75%, >80%, >85%, >90%, >95%, or 100% at day 8 of cycle 1.

24. The antibody for use according to claim 5, wherein the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100% at day 8 of cycle 1.

25. The antibody for use according to claim 5, wherein the antibody leads to a change from baseline anti-PLA2R titer between 25% and 80%.

26. The antibody for use according to claim 25, wherein the antibody leads to a change from baseline anti-PLA2R titer between 25% and 80% over 4 weeks of treatment.

27. The antibody for use according to claim 5, wherein the antibody leads to a change from baseline anti-PLA2R titer of >80%.

28. The antibody for use according to claim 27, wherein the antibody leads to a change from baseline anti-PLA2R titer of >80% over 4 weeks of treatment.

29. The antibody for use according to claim 5, wherein the antibody does not increase the anti-PLA2R titer over 4 weeks of treatment.

30. The antibody for use according to any one of the preceding claims, wherein said antibody comprises a variable heavy chain of the sequence QVQLVESGGGLVQPGGSLRLSCAASGFTFSSYYMNWVRQAPGKGLEWVSGISGDPSNTY YADSVKGRFTISRDNSKNTLYLQMNSLRAEDTAVYYCARDLPLVYTGFAYWGQGTLVTVSS (SEQ ID NO: 7) and a variable light chain of the sequence DIELTQPPSVSVAPGQTARISCSGDNLRHYYVYWYQQKPGQAPVLVIYGDSKRPSGIPERFS GSNSGNTATLTISGTQAEDEADYYCQTYTGGASLVFGGGTKLTVLGQ (SEQ ID NO: 8).

31. The antibody for use according to any of the preceding claims, wherein said antibody comprises an IgG1 Fc region.

32. The antibody for use according to any of the preceding claims, wherein said antibody is formulated for administration in combination with 650 mg to 1,000 mg of oral paracetamol.

33. The antibody for use according to any of the preceding claims, wherein said antibody is formulated for administration in combination with 25 mg to 50 mg of oral or intravenous diphenhydramine.

34. The antibody for use according to any of the preceding claims, wherein said antibody is formulated for administration in combination with 100 mg of intravenous methylprednisolone, or an equivalent thereof.

35. Felzartamab or a biosimilar of felzartamab for use in the treatment of a subject with anti-PLA2R-mediated membranous nephropathy, wherein felzartamab or a biosimilar of felzartamab is administered at a fixed dose of 650 mg, 975 mg, 1300 mg or 1625 mg, corresponding to a body weight range of <50 kg, 50.5 to 70 kg, 70.5 to 90 kg, >90.5 kg, respectively, and wherein felzartamab or a biosimilar of felzartamab is administered at an interval of i) day 1, day 8, day 15, day 29, and day 57 representing a 3 month treatment period; or ii) day 1 and day 15 representing a 1 month treatment period, and optionally with a re-treatment 3 or 5 month after end of the preceding treatment period, and wherein felzartamab or a biosimilar of felzartamab is administered intravenously.

36. A method for treating autoantibody-mediated membranous nephropathy, comprising administering to a subject in need thereof a therapeutically effective amount of an antibody specific for CD38 comprising an HCDR1 region of sequence GFTFSSYYMN (SEQ ID NO: 12) or SYYMN (SEQ ID NO: 1), an HCDR2 region of sequence GISGDPSNTYYADSVKG (SEQ ID NO: 2), an HCDR3 of sequence DLPLVYTGFAY (SEQ ID NO: 3), an LCDR1 region of sequence SGDNLRHYYVY (SEQ ID NO: 4), an LCDR2 region of sequence GDSKRPS (SEQ ID NO: 5), and an LCDR3 region of sequence QTYTGGASL (SEQ ID NO: 6), wherein said antibody is administered at a fixed dose level.

37. The method according to claim 36, wherein the autoantibody-mediated membranous nephropathy is an anti-PLA2R-positive membranous nephropathy.

38. The method according to claim 36 or claim 37, wherein said antibody is administered at a fixed dose corresponding to a body weight range of the subject.

39. The method according to claim 38, wherein said antibody is administered at a fixed dose of 650 mg, 975 mg, 1300 mg or 1625 mg, corresponding to a body weight range of <50 kg, 50.5 to 70 kg, 70.5 to 90 kg, >90.5 kg, respectively.

40. The method according to claim 36-37, wherein said antibody is administered at a fixed dose of 16 mg/kg.

41. The method according to any of the claims 36-40, wherein the antibody is administered in the initial 3 weeks once weekly (QW).

42. The method according to any of the claims 36-41, wherein the antibody is administered once every two weeks (q2w).

43. The method according to any of the claims 36-42, wherein said antibody is administered at the interval of i) day 1, day 8, day 15, day 29, and day 57 representing a 3 month treatment period or ii) day 1 and day 15 representing a 1 month treatment period.

44. The method according to claim 43, further comprising re-treatment 3 or 5 months after end of the preceding treatment period.

45. The method according to any one of claims 36-40, wherein the antibody is administered at the interval of day 1, day 8, day 15, and day 22, followed by day 29, day 57, day 85, day 113, and day 141 in a treatment period of 24 weeks.

46. The method according to any one of claims 36-40, wherein said antibody is administered at two doses in a treatment interval of 85 days.

47. The method according to any one of claims 36-40, wherein said antibody is administered at five doses in a treatment interval of 85 days.

48. The method according to any one of claims 36-40, wherein said antibody is administered at nine doses in a treatment interval of 24 weeks.

49. The method according to any one of claims 36-40, wherein said antibody is administered at nine doses in a treatment interval of 141 days.

50. The method according to claim 40, wherein said antibody is administered once weekly during the first month of treatment and then once every 4 weeks following the first month.

51. The method according to any of the claims 36-50, wherein said antibody is administered intravenously.

52. The method according to claim 51, wherein the antibody is administered intravenously over a period of two hours.

53. The method according to any of the claims 36-52, wherein the subject has a serum level of anti-PLA2R antibodies of ≥50 RU/mL, ≥100 RU/mL, ≥150 RU/mL, ≥200 RU/mL, ≥250 RU/mL, ≥300 RU/mL at screening.

54. The method according to claim 53, wherein the subject has a serum level of anti-PLA2R antibodies of ≥150 RU/mL.

55. The method according to any of the claims 36-54, wherein the subject has a urine protein:creatinine ratio (UCPR; g/g)) of ≥3.0 g/g, ≥4.0 g/g, ≥5.0 g/g, ≥6.0 g/g, or ≥7.0 g/g.

56. The method according to any of the claims 36-55, wherein the subject has a proteinuria of ≥3.5 g/24 h, ≥4.0 g/24 h, ≥4.5 g/24 h or ≥5.0 g/24 h from a 24-hour urine screening.

57. The method according to any of the claims 36-56, wherein the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100%.

58. The method according to claim 57, wherein the subject has serum anti-PLA2R antibodies of ≥50 RU/mL, and the reduction of anti-PLA2R autoantibody titer compared to baseline is >10%, >15%, >20% >25%, >30%, >35%, >40%, >45%, >50%, >55%, >60%, >65%, >70%, >75%, >80%, >85%, >90%, >95%, or 100% at day 8 of cycle 1.

59. The method according to claim 40, wherein the antibody leads to a change from baseline anti-PLA2R titer of >10%, >15%, >20% >25%, >30%, >35%, >40%, >45%, >50%, >55%, >60%, >65%, >70%, >75%, >80%, >85%, >90%, >95%, or 100% at day 8 of cycle 1.

60. The method according to claim 40, wherein the antibody leads to a change from baseline anti-PLA2R titer between 25% and 80%.

61. The method according to claim 60, wherein the antibody leads to a change from baseline anti-PLA2R titer between 25% and 80% over 4 weeks of treatment.

62. The method according to claim 40, wherein the antibody leads to a change from baseline anti-PLA2R titer of >80%.

63. The method according to claim 62, wherein the antibody leads to a change from baseline anti-PLA2R titer of >80% over 4 weeks of treatment.

64. The method according to claim 40, wherein the antibody does not increase the anti-PLA2R titer over 4 weeks of treatment.

65. The method according to any one of the claims 36-64, wherein said antibody comprises a variable heavy chain of the sequence QVQLVESGGGLVQPGGSLRLSCAASGFTFSSYYMNWVRQAPGKGLEWVSGISGDPSNTY YADSVKGRFTISRDNSKNTLYLQMNSLRAEDTAVYYCARDLPLVYTGFAYWGQGTLVTVSS (SEQ ID NO: 7) and a variable light chain of the sequence DIELTQPPSVSVAPGQTARISCSGDNLRHYYVYWYQQKPGQAPVLVIYGDSKRPSGIPERFS GSNSGNTATLTISGTQAEDEADYYCQTYTGGASLVFGGGTKLTVLGQ (SEQ ID NO: 8).

66. The method according to any of the claims 36-65, wherein said antibody comprises an IgG1 Fc region.

67. The method according to any of the claims 36-66, wherein said antibody administered in combination with 650 mg to 1,000 mg of oral paracetamol.

68. The method according to any of the claims 36-67, wherein said antibody is administered in combination with 25 mg to 50 mg of oral or intravenous diphenhydramine.

69. The method according to any of the claims 36-68, wherein said antibody is administered in combination with 100 mg of intravenous methylprednisolone, or an equivalent thereof.

70. A method for treating anti-PLA2R-mediated membranous nephropathy, comprising administering to a subject in need thereof a therapeutically effective amount of felzartamab or a biosimilar of felzartamab, wherein felzartamab or a biosimilar of felzartamab is administered at a fixed dose of 650 mg, 975 mg, 1300 mg or 1625 mg, corresponding to a body weight range of <50 kg, 50.5 to 70 kg, 70.5 to 90 kg, >90.5 kg, respectively, and wherein felzartamab or a biosimilar of felzartamab is administered at an interval of i) day 1, day 8, day 15, day 29, and day 57 representing a 3 month treatment period; or ii) day 1 and day 15 representing a 1 month treatment period, and optionally with a re-treatment 3 or 5 month after end of the preceding treatment period, and wherein felzartamab or a biosimilar of felzartamab is administered intravenously.
